(19) United States
(12) Patent Application Publication
(10) Pub. No.: US 2013/0131088 A1
(43) Pub. Date: May 23, 2013

(54) TREATING CANCER WITH STATINS AND COMPOUNDS HAVING DIPYRIDAMOLE ACTIVITY

(71) Applicants: Linda Penn, Toronto (CA); Aaron Schimmer, Thornhill (CA); Aleksandra Pandyra, Toronto (CA)

(72) Inventors: Linda Penn, Toronto (CA); Aaron Schimmer, Thornhill (CA); Aleksandra Pandyra, Toronto (CA)

(73) Assignee: UNIVERSITY HEALTH NETWORK, Toronto, ON (CA)

(21) Appl. No.: 13/522,242

(22) PCT Filed: Jan. 13, 2011
(86) PCT No.: PCT/CA11/00026

§ 371 Date: Jul. 13, 2012

Related U.S. Application Data
(60) Provisional application No. 61/294,691, filed on Jan. 13, 2010.
(60) Provisional application No. 61/294,685, filed on Jan. 13, 2010.

(51) Int. Cl. A61K 31/519; A61K 31/40; A61K 31/405; A61K 31/352; A61K 31/215; A61K 31/47; A61K 31/4709; A61K 31/351; A61K 31/505

(52) U.S. Cl. CPC A61K 31/519; A61K 31/351; A61K 31/40; A61K 31/405; A61K 31/505; A61K 31/215; A61K 31/47; A61K 31/4709; A61K 31/352
USPC 514/262.1; 514/460; 514/423; 514/419; 514/275; 514/546; 514/311; 514/312; 514/455

(57) ABSTRACT

The disclosure pertains to methods of treating a cancer comprising administering to a subject in need thereof an effective amount of a statin in combination with an effective amount of a dipyridamole and/or a compound that has dipyridamole activity.

### RELATED APPLICATIONS

This is a Patent Cooperation Treaty Application which claims the benefit of 35 U.S.C. 119 based on the priority of corresponding U.S. Provisional Patent Application Nos. 61/294,685 and 61/294,691, both filed Jan. 13, 2010, each of which is herein incorporated by reference in its entirety.

### FIELD OF THE DISCLOSURE

The disclosure relates to methods and compositions for the treatment of cancer and particularly to methods and compositions comprising two compounds for the treatment of hematological malignancies such as acute myeloid leukemia (AML) or multiple myeloma (MM) in a subject.

### BACKGROUND OF THE DISCLOSURE

The statin family of drugs consists of seven unique compounds that function similarly as potent competitive inhibitors of hydroxymethylglutaryl coenzyme A reductase (HMGCR) activity, yet they differ in their chemical and pharmacological properties (FIGS. 1 and 2) (1-4). By inhibiting HMGCR, statins block production of the immediate product, mevalonate (MVA), and many end-products of this pathway required for a wide range of essential cellular functions. These include: sterols, such as cholesterol, involved in membrane integrity and steroid production; ubiquinone, involved in electron transport and cell respiration; farnesyl and geranylgeranyl isoprenoids, important for targeting proteins such as the Ras family to membranes; dolichol, required for glycoprotein synthesis; and isopentenyladenine, essential for protein synthesis.

Depletion of MVA pathway components in normal cells triggers a feedback response that ensures cells quickly up-regulate HMGCR and restore the MVA pathway (FIG. 1) (5-7). Recent evidence suggests that deregulation of the MVA pathway contributes to cancer and, importantly, that blocking the rate-limiting enzyme of the MVA pathway with statins can effectively stop tumour growth (6). For example, providing exogenous MVA increases the growth of human cancer xenografts (8) and, conversely, exposure to statins blocks proliferation of cancer cell lines (9, 10). Indeed, the anti-proliferative effects of statins are tumour-selective and do not cause collateral damage to normal cells both in tissue culture and animal models.

Epidemiological as well as prospective studies addressing statin use, cancer incidence and survival show mixed results, with some reporting inverse associations with as much as 50% reduction in cancer risk amongst statin users and others reporting no association (11-17). Interestingly, the latter reports have been criticized for insufficient control of covariates, including the type and duration of statin use as well as the specific cancer sub-type under evaluation. More recent epidemiological studies that accommodated these covariates show that statins confer a lower incidence of cancer, a lower grade and stage, and decreased risk of recurrence (18-25).

The anti-cancer properties of statins have already prompted fourteen reported Phase I and I/II clinical trials (26-39). In the early dose-finding studies the impact on tumour growth was not a primary focus of evaluation, but stable disease and partial or complete remissions were evident in some, but not all, patients (26, 27, 30, 31, 34, 35, 37, 40). Indeed, low, cholesterol-lowering-like doses administered for prolonged duration was associated with the control of tumour burden with minimal side-effects (41a and b). It remains unclear why certain patients are sensitive to the anti-cancer properties of statins while others remain insensitive, however, recent trials suggest overall efficacy is increased when statins are used in combination therapy (35, 36). This is consistent with the use of drug cocktails to target multiple deregulated pathways essential for tumour growth and survival. Surprisingly, however, of the 20 ongoing clinical cancer trials with statins (42), the particular statin and cohort of patients under investigation appear to be arbitrarily chosen. Taken together, evidence from clinical trials focused on statins as anti-cancer agents indicates that responses are possible and combination therapies show maximal efficacy.

### SUMMARY OF THE DISCLOSURE

An aspect of the disclosure includes a method of treating a cancer or a precancerous disorder comprising administering to a subject in need thereof a statin in combination with a compound having a dipyridamole activity.

In an embodiment, the dipyridamole activity is PKA activation and the compound with dipyridamole activity is a PKA activator. In an embodiment, the dipyridamole activity is HMGCR expression inhibition and the compound with dipyridamole activity is a HMGCR expression inhibitor. In an embodiment the compound is a PKA activator and HMGCR expression inhibitor. Accordingly, in an embodiment, the method comprises administering to a subject in need thereof a statin in combination with a PKA activator and/or HMGCR expression inhibitor.

In an embodiment, the compound with dipyridamole activity is dipyridamole.

In an embodiment, the method comprises administering to a subject in need thereof, an effective amount of a statin, in combination with an effective amount of dipyridamole.

In another embodiment, the method comprises inducing cell death in a cancer cell or a precancerous cell comprising contacting the cell with a statin in combination with dipyridamole and/or a compound having dipyridamole activity.

In a further embodiment, the cancer is selected from a hematological cancer and a solid tumor, such as colorectal cancer, skin cancer, prostate cancer, hepatocellular carcinoma (HCC), breast or lung cancer. In another embodiment, the precancerous disorder is a hematological precancerous disorder, such as monoclonal gammopathy of undetermined significance (MGUS) or myelodysplastic syndrome (MDS).

In another embodiment, the hematological cancer is a leukemia, myeloma or a lymphoma.

In yet a further embodiment, the leukemia is selected from acute myeloid leukemia (AML), acute lymphocytic leukemia (ALL) and chronic myelogenous leukemia (CML).

Another aspect includes a composition comprising a statin and compound having dipyridamole activity and optionally a suitable carrier or vehicle.

In an embodiment, the composition comprises a statin and dipyridamole and optionally a suitable carrier or vehicle.

In an embodiment, the compound or compound having a dipyridamole activity is selected from a PDE inhibitor such as a PDE3 inhibitor, an adenylate cyclase activator, a cell permeable cAMP analogue. In an embodiment, the compound is selected from cilostazol, db-cAMP and forskolin. In an embodiment, the statin comprises a group of the formula Ia or Ib is or is selected from lovastatin, simvastatin, atorvastatin, fluvastatin, rosuvastatin, pravastatin, and pitavastatin.

A further aspect includes a kit comprising a statin and instructions for administering in combination with a compound having dipyridamole activity.

In an embodiment, the kit comprises a statin and/or a compound having dipyridamole activity and instructions for administering in combination with the other.

Yet a further aspect includes a pack comprising a statin and dipyridamole and/or a compound with dipyridamole activity and instructions for administering for example a daily dose for the treatment of a cancer such as a solid cancer, a hematological cancer and/or a precancerous disorder such as a hematological precancerous disorder.

Other features and advantages of the present disclosure will become apparent from the following detailed description. It should be understood, however, that the detailed description and the specific examples while indicating preferred embodiments of the disclosure are given by way of illustration only, since various changes and modifications within the spirit and scope of the disclosure will become apparent to those skilled in the art from this detailed description.

### BRIEF DESCRIPTION OF THE DRAWINGS

FIG. 1. Statins block the mevalonate pathway by inhibiting the rate-limiting enzyme, HMGCR. Statins compete with the natural substrate, HMG-CoA, for the active site of HMGCR. Mva, the product of the reaction, is the precursor to many critical cellular end-products essential for cell proliferation and survival. The anti-proliferative activity of statins on tumour cells is blocked with excess MVA or geranylgeranyl pyrophosphate (GGPP), suggesting that statins block one or more signaling pathways that are critical for survival of the cancer cell.

FIG. 2. Structure and therapeutically-relevant properties of 7 of the available statins. Boxes highlight the statins' active open ring that mimics the reaction intermediate formed during the HMGCR-catalyzed reaction.

FIG. 3. A chemical screen (A) in the KMS11 Multiple Myeloma (MM) cells identified dipyridamole (B) as potentiating the cytotoxic effect of atorvastatin as ascertained by MTS activity following compound exposure for 72 hours. A hundred compounds from the chemical library (see text) were assayed at varying concentrations (5-50 μM). The concentrations, as tested in a variety of AML cell lines, were intended to have a 20% killing effect upon treatment with each individual drug alone.

FIG. 4. Dipyridamole synergized with atorvastatin (A,C) and fluvastatin (B,D) in several MM (A,B) and AML (C,D) cell lines. Drug treatments were performed individually or in a fixed ratio for 48 hours. Synergy was assessed using Chou and Talays combination index.

FIG. 5. The combination of dipyridamole and atorvastatin induced apoptosis in the KMS11 cell line as ascertained by fixed PI (A), TUNEL (B) and PARP cleavage (C) in an MVA dependant manner. Assays were performed following 48 hrs of exposure at the indicated concentrations (A=atorvastatin, D=dipyridamole, MVA=mevalonate, concentrations (μM) are indicated in brackets). n=3-4, *p<0.05, a one-way ANOVA with a post Tukey test.

FIG. 6. The combination of dipyridamole and atorvastatin induced apoptosis in the AML-3 cell line as ascertained by fixed PI (A) TUNEL (B) and PARP cleavage (C) in an MVA dependant manner. Assays were performed following 48 hrs of exposure at the indicated concentrations (A=atorvastatin, D=dipyridamole, MVA=mevalonate, concentrations (μM) are indicated in brackets). n=3-4, *p<0.05, a one-way ANOVA with a post Tukey test.

FIG. 7. The combination of dipyridamole and statins was effective at induceing apoptosis in primary AML patient samples (A) as ascertained by Annexin V (AV) and propidium ioidide (PI) dual staining ( ), representative histograms shown from patient 1 (B). Assay was performed following 48 hrs of exposure at the indicated concentrations (A=atorvastatin, D=dipyridamole, F=Fluvastatin), concentrations (μM) are indicated in the bar graph. % Apoptosis \ represents the summation of AV+/PI− (early apoptosis) and AV+/PI+ (late apoptosis). AV−/PI−=live cells, AV+/PI−=early apoptosis, AV+/PI+=late apoptosis, AV−/PI+=necrosis. The combination was not toxic to normal cells (PBSC1) at the highest concentrations assessed for the primary AML patient samples.

FIG. 8. Atorvastatin treatment, p.o. (oral delivery) three times a week for 37 days initiated 2 days post KMS11-luc (KMS11 cells stably expressing luciferase) injection (8 million cells/mouse inoculated i.v. via the tail) significantly reduced tumor burden (A) and prolonged survival (B) in the early stage MM disease model (n=7-8 mice/group) representative animals shown in (C). Lower doses of atorvastatin (p.o. 3 times a week) as well as a 60 mg/kg DP dose (p.o. 5 times a week) had no significant effect, (n=3-5 mice/group) when administered alone (D).

FIG. 9. Dipyridamole potentiated the anti-cancer effects of statins (A) and synergized with statins (B) in several breast cancer and lung cell lines. Drug treatments were performed individually or in a fixed ratio for 72 hours. Synergy was assessed using Chou and Talays combination index. (DP=dipyridamole), n=1-4. Atorvastatin was used in A549's and fluvastatin the breast cancer cell lines to asses potentiation and synergy.

FIG. 10. Dipyridamole is an agent known to elicit several molecular effects, amongst them phosphodiesterase (PDE) inhibition. Addition of a cell-permeable analog of cAMP, dibutyryl-cAMP (db-cAMP 0.25 mM) in combination with atorvastatin recapitulated the DP-atorvastatin apoptotic effects (A) 48 hours post treatment in the AML-3 cell line. Cilostazol, a selective PDE3 inhibitor, also potentiated the cytotoxic effects of statins in AML-3 (B) and AML-2 (C) and KMS11 (D) cell lines as did forskolin an adenylate cyclase activator in the AML-3 cell line (E) (For=forskolin, F=Fluvastatin, concentrations (μM) indicated). db-cAMP, DP and forskolin increase CREB phosphorylation 2 hours post-treatment in the AML-3 cell line as demonstrated in the representative immunoblot (F) quantified in (G) using secondary antibodies labeled with IRDye infrared dyes(LI-COR). n=2-3*p<0.05

FIG. 11. The combination of statins and dipyridamole prevented the upregulation of HMGCR mRNA in LP1 mM cells (A) and primary AML patient samples (B,C). RNA from primary AML and LP1 cells was harvested 24 and 16 hours post compound treatment respectively using TRIZOL reagent. HMGCR mRNA levels were assessed using real-time PCR (SYBR Green). Ator.=Atorvastatin, F or Fluva=Fluvastatin, DP=Dipyridamole, concentrations (μM) depicted in the bar graphs. For example, Fluva 2 indicates a fluvastatin concentration of 2 μM.

FIG. 12. Combination tests.

### DETAILED DESCRIPTION OF THE DISCLOSURE

### I. Definitions

The term “statins” as used herein refers to the general class of compounds that are known inhibitors of HMGCR. In an embodiment the statin will have, within its structure, a moiety that mimics the reaction intermediate formed during the HMGCR catalyzed reaction. In a further embodiment this moiety is a group of the formula Ia or Ib:

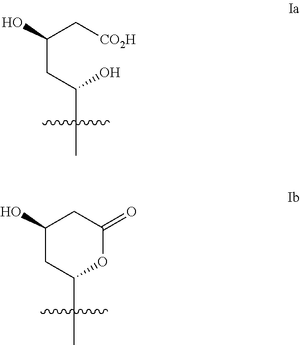 [SMILES: *[C@@H](O)C[C@@H](O)CC(=O)O ; *[C@@H]1C[C@@H](O)CC(=O)O1]

In a further embodiment, the statin is in the form of a neutral compound or as pharmaceutically acceptable salt. In another embodiment, the statin, or salt thereof, is in the form of a solvate or prodrug thereof. In a further embodiment, the statin may be a mixture of two or more statins, or pharmaceutically acceptable salts, solvates or prodrugs thereof. In another embodiment, the statin is selected from lovastatin (Mevacor™), simvastatin (Zocor™), atorvastatin (Lipitor™), fluvastatin (Lescol™), rosuvastatin (Crestor™), pravastatin (Pravachol™), or pitavastatin (LIVALO®), or a pharmaceutically acceptable salt, solvate or prodrugs thereof, or a mixture thereof. In yet another embodiment, the statin is lovastatin, atorvastatin, fluvastatin, or pitavastatin or a pharmaceutically acceptable salt, solvate or prodrugs thereof, or a mixture thereof. The term “lovastatin” as used herein means a compound having the structure:

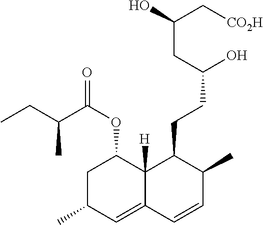 [SMILES: CC[C@H](C)C(=O)O[C@H]1C[C@@H](C)C=C2C=C[C@H](C)[C@H](CC[C@@H](O)C[C@@H](O)CC(=O)O)[C@H]21]

or a pharmaceutically acceptable salt, solvate or prodrug thereof as well as mixtures thereof.

The term “simvastatin” as used herein means a compound having the structure:

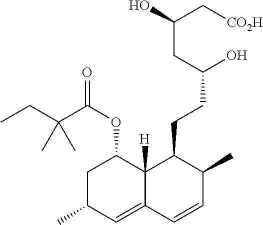 [SMILES: CCC(C)(C)C(=O)O[C@H]1C[C@@H](C)C=C2C=C[C@H](C)[C@H](CC[C@@H](O)C[C@@H](O)CC(=O)O)[C@H]21]

or a pharmaceutically acceptable salt, solvate or prodrug thereof as well as mixtures thereof.

The term “atorvastatin” as used herein means a compound having the structure:

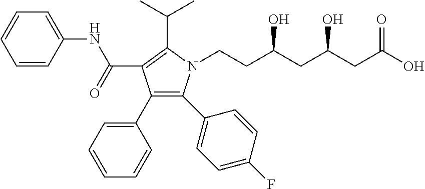 [SMILES: CC(C)c1c(C(=O)Nc2ccccc2)c(-c2ccccc2)c(-c2ccc(F)cc2)n1CC[C@@H](O)C[C@@H](O)CC(=O)O]

or a pharmaceutically acceptable salt, solvate or prodrug thereof as well as mixtures thereof.

The term “fluvastatin” as used herein means a compound having the structure:

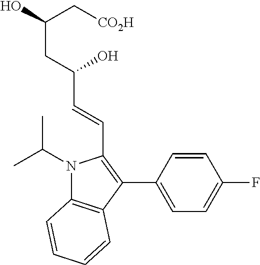 [SMILES: CC(C)n1c(/C=C/[C@@H](O)C[C@@H](O)CC(=O)O)c(-c2ccc(F)cc2)c2ccccc21]

or a pharmaceutically acceptable salt, solvate or prodrug thereof as well as mixtures thereof.

The term “rosuvastatin” as used herein means a compound having the structure:

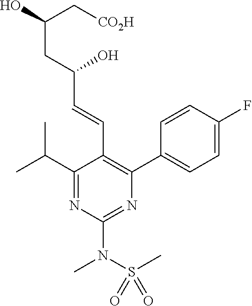 [SMILES: CC(C)c1nc(N(C)S(C)(=O)=O)nc(-c2ccc(F)cc2)c1/C=C/[C@@H](O)C[C@@H](O)CC(=O)O]

or a pharmaceutically acceptable salt, solvate or prodrug thereof as well as mixtures thereof.

The term “pravastatin” as used herein means a compound having the structure:

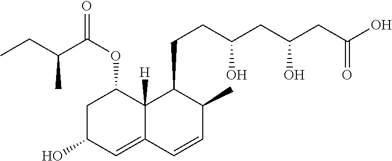 [SMILES: CC[C@H](C)C(=O)O[C@H]1C[C@@H](O)C=C2C=C[C@H](C)[C@H](CC[C@@H](O)C[C@@H](O)CC(=O)O)[C@H]21]

or a pharmaceutically acceptable salt, solvate or prodrug thereof as well as mixtures thereof.

The term “pitavastatin” as used herein means a compound having the structure:

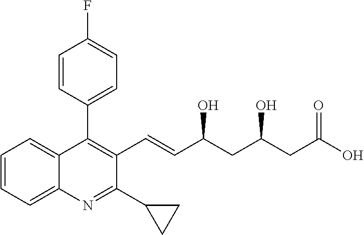 [SMILES: O=C(O)C[C@H](O)C[C@H](O)/C=C/c1c(C2CC2)nc2ccccc2c1-c1ccc(F)cc1]

or a pharmaceutically acceptable salt, solvate or prodrug thereof as well as mixtures thereof.

The term “dipyridamole” as used herein means a compound having the structure:

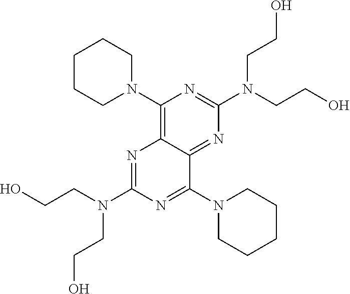 [SMILES: OCCN(CCO)c1nc(N2CCCCC2)c2nc(N(CCO)CCO)nc(N3CCCCC3)c2n1]

or a pharmaceutically acceptable salt, solvate or prodrug thereof as well as mixtures thereof. Dipyridamole is also known as RZ-8, Persantin, Dipyridamine, Dipyridamol, Dipuydamine and sold for example under the brand name Persantine™.

The term “dipyridamole analogue” as used herein means a dipyridamole compound that is modified chemically that retains or has an enhanced PKA inducing dipyridamole activity, and/or HMGCR expression inhibitor dipyridamole activity. Analogues include for example analogues with these properties described in Resistance-modifying agents. 11.(1) Pyrimido[5,4-d]pyrimidine modulators of antitumor drug activity; Synthesis and structure-activity relationships for nucleoside transport inhibition and binding to alpha1-acid glycoprotein, Curtin N J, Barlow H C, Bowman K J, Calvert A H, Davison R, Golding B T, Loughlin P J, Newell D R, Smith P G, Griffin R J. J Med. Chem. 2004 Sep. 23; 47(20):4905-22 and Synthesis, flow cytometric evaluation, and identification of highly potent dipyridamole analogues as equilibrative nucleoside transporter 1 inhibitors. J Med. Chem. 2007 Aug. 9; 50(16):3906-20. Epub 2007 Jul. 18. Others analogues include ones described in Lin, W. and Buolamwini J K (2007) Synthesis, flow cytometric evaluation, and identification of highly potent dipyridamole analogues as equilibrative nucleoside transporter 1 inhibitors J Med Chem 50:3906-20, Kadatz R. The Pharmacology of 2,6-bis(diethanolamino)-4,8-dipiperidino-pyrimido-(5,4-d)-pyrimidine, a new compound with coronary dilatory properties. Arzneimittelforsch 1959; 9:39-45. [PubMed: 13628477]; Fischer, F G.; Roch, W.; Roch, J.; Kottler, A. Substituted Pyrimido-[5,4-d] Pyrimidines. U.S. Pat. No. 3,031,450. 1962; Roch, J.; Beisenherz, G.; an der Riss, B. Basic-Substituted Pyrimido-[5,4-d]-Pyrimidines. U.S. Pat. No. 3,074,928, 1963; Kaminsky D, Lutz W B, Lazarus S. Some Congeners and Analogs of Dipyridamole. J Med Chem 1966; 9:610-612 [PubMed:5968035] and Roch, J.; Erich, M.; Narr, B.; Machleidt, H. S African pat 6,905,504. 1970, each of which are herein incorporated by reference.

The term “dipyridamole activity” as used herein refers to one or more biological activities of dipyridamole selected from dipyridamole's biological activity as a PKA activator as an inhibitor of phosphodiesterases (PDEs) involved in PKA activation, and HMGCR expression (e.g. HMGCR statin induced upregulation is inhibited by dipyridamole when administered to cells in combination with a statin). Inhibition of certain PDEs activates for example the cAMP-dependent protein kinase A (PKA). PKA, involved in a number of cellular processes, is a member of the serine-threonine protein kinase superfamily. PKA activation results in the subsequent translocation of the PKA free catalytic subunit into the nucleus where phosphorylation of nuclear substrates largely composed of transcription factors of the cAMP response element binding (CREB) family occurs. Activation of PKA holoenzymes, which are inactive heterotetramers, is mediated by binding of two cAMP molecules to PKA regulatory (R) subunits resulting in the release and activation of the catalytic subunits. The intracellular levels of cAMP are tightly regulated by its synthesis and degradation mediated by adenylate cyclases (ACs) and cAMP phosphodiesterases (PDEs) respectively. Conversion of ATP into cAMP by ACs raises intracellular cAMP levels thereby activating PKA; conversely degradation of cAMP by PDEs limits PKA activation. Further attempts to modulate PKA activity can occur through inhibition or activation of the aforementioned enzymes. Inhibition of PDEs, using agents such as for example cilostazol, a 3-type PDE (PDE3) inhibitor or dipyridamole, a multi PDE inhibitor, can increase cAMP levels and activate PKA. Activation of AC by activators such as forskolin, will also have similar consequences. It is demonstrated herein that dipyridamole can inhibit HMGCR upregulation in combination with statins and trigger cell death. Accordingly, dipyridamole activity includes for example PKA activation activity, PDE inhibitor activity, such as PDE3 inhibitor activity, and HMGCR expression inhibitor activity.

The term “a compound having a dipyridamole activity” refers to a compound such as dipyridamole and/or dipyridamole analogue that alone or in combination with a statin activates PKA and/or inhibits HMGCR expression and includes for example dipyridamole and structurally unrelated molecules with PKA inducing activity and/or HMGCR expression inhibiting activities similar to (or enhanced compared to) dipyridamole. For example, dipyridamole is an inhibitor of adenosine uptake and PDEs. Cilostazol is also an inhibitor of adenosine uptake and PDE3 resulting in increased cAMP levels such that cilostazol is a compound having a dipyridamole activity. A compound having a dipyridamole activity includes a PKA activator, including for example a PDE3 inhibitor, and an AC activator, and/or a HMGCR expression inhibitor.

The term “PKA activator” as used herein means a compound that activates the kinase activity of cAMP dependent protein kinase A and leads to CREB phosphorylation. Examples include adenylate cyclase activators such as forskolin, cell permeable cAMP anologues such as db-cAMP, and PDE inhibitors including PDE3 inhibitors such as cilostazol as well as pharmaceutically acceptable salts, solvates or prodrugs thereof as well as mixtures thereof.

The term “PDE inhibitor” as used herein means a compound that inhibits a PDE that increases cAMP levels and induces PKA activation. For example, inhibition of PDE1, PDE2, PDE4 (Rolipram), PDE7, PDE8 and PDE10 (Papaverine) and PDE3 (Cilostazol) all degrade cAMP and their subsequent inhibition will cause in increased intracellular levels of cAMP.

The term “PDE3 inhibitor” as used herein refers to a compound that inhibits the phosphodiesterase activity of PDE3, including for example cilostazol, aminone or inaminone, milrinone and enoximone as well as pharmaceutically acceptable salts, solvates or prodrugs thereof as well as mixtures thereof.

The term to “inhibit” or “suppress” or “reduce” a function or activity, such as PDE, means any detectable or measurable reduction in the function or activity (e.g. PKA activation) when compared to otherwise same conditions, except for a condition or parameter of interest, or alternatively, as compared to another condition.

The term “HMGCR expression inhibitor” as used herein refers to a compound that when used alone or in combination for example with a statin decreases HMGCR expression and/or inhibits statin induced HMGCR feed back upregulation of HMGCR mRNA expression. It is demonstrated herein, that dipyridamole when added in combination with statin inhibits statin induced HMGCR upregulation. Accordingly, HMGCR expression inhibitors includes compounds that inhibit HMGCR expression alone or in combination with statin and includes for example dipyridamole.

The term “cilostazol” as used herein means a compound having the structure:

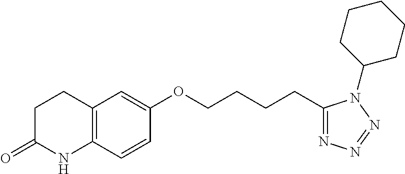 [SMILES: O=C1CCc2cc(OCCCCc3nnnn3C3CCCCC3)ccc2N1]

or a pharmaceutically acceptable salt, solvate or prodrug thereof as well as mixtures thereof. Cilostazol is also known as cilostazolum and sold under the brand names Pletaal™ and Pletal™.

The term “mixture” as used herein, means a composition comprising two or more of compounds. In an embodiment a mixture is a mixture of two or more distinct compounds. In a further embodiment, when a compound is referred to as a “mixture”, this means that it can comprise two or more “forms” of the compounds, such as, salts, solvates, prodrugs or, where applicable, stereoisomers of the compound in any ratio. A person of skill in the art would understand that a compound in a mixture can also exist as a mixture of forms. For example, a compound may exist as a hydrate of a salt or as a hydrate of a salt of a prodrug of the compound. All forms of, the compounds disclosed herein, in and ratio or combination, are within the scope of the present disclosure.

The term “potentiates” as used herein means the compound significantly increases statin induced cell killing and significantly shifts the statin dose-response curve and IC50 (the half-maximal inhibitory concentration). Potentiates is for example greater than additive.

The term “synergistic” as used herein means the enhanced or magnified effect of a combination on at least one property compared to the additive individual effects of each component of the combination. For example, compounds that induce cell death by the same mechanism, e.g. would not be expected to have more than additive effect. Synergism can be assessed and quantified for example by analyzing the Data by the Calcusyn median effect model where the combination index (CI) indicates synergism (CI<0.9), additively (CI=0.9-1.1) or antagonism (CI>1.1). CIs of <0.3, 0.3-0.7, 0.7-0.85, 0.85-0.90, 0.90-1.10 or >1.10 indicate strong synergism, synergism, moderate synergism, slight synergism, additive effect or antagonism, respectively. The CI is the statistical measure of synergy.

The term “cell death” as used herein includes all forms of cell death including necrosis and apoptosis.

As used herein, “contemporaneous administration” and “administered contemporaneously” means that the statin and dipyridamole and/or a compound with dipyridamole activity, are administered to a subject such that they are each biologically active in the subject at the same time. The exact details of the administration will depend on the pharmacokinetics of the substances in the presence of each other, and can include administering one substance for example within 24, 48 or 72 hours of administration of another, if the pharmacokinetics are suitable. Designs of suitable dosing regimens are routine for one skilled in the art. In particular embodiments, two substances will be administered substantially simultaneously, i.e. within minutes of each other, or in a single composition that comprises both substances.

The term “combination therapy” or “in combination with” as used herein means two or more substances, for example a statin and dipyridamole, and/or for example a statin and dipyridamole and/or a compound having dipyridamole activity, in combination with molecular targeted therapeutics, chemotherapeutics and radiation, are administered to a subject over a period of time, contemporaneously or sequentially e.g. the substances are administered at the same time or at different times within the period of time in a regimen that will provide beneficial effects of the drug combination, at similar or different intervals. For example, the combination therapy is intended to embrace co-administration, in a substantially simultaneous manner such as in a single dosage form e.g. a capsule, having a fixed ratio of active ingredients or in multiple, separate dosage forms, e.g. capsules, for each substance. The compounds may or may not be biologically active in the subject at the same time. As an example, a first substance is administered weekly, and a second substance administered every other week for a number of weeks. The exact details of the administration will depend on the pharmacokinetics of the two substances. Designs of suitable dosing regimens are routine for one skilled in the art.

As used herein, the phrase “dosage form” refers to the physical form of a dose for example comprising statin and/or dipyridamole and/or a compound having dipyridamole activity such as a dipyridamole anlalogue, and includes without limitation tablets, including enteric coated tablets, caplets, gelcaps, capsules, ingestible tablets, buccal tablets, troches, elixirs, suspensions, syrups, wafers, liposomal formulations and the like. The dosage form may be solid or liquid. Liposomal formulations, can for example be used to administer multiple compounds at fixed ratios.

As used herein, the phrase “effective amount” or “therapeutically effective amount” means an amount effective, at dosages and for periods of time necessary to achieve the desired result. For example in the context or treating a cancer such as a hematological cancer, an effective amount is an amount that for example induces remission, reduces tumor burden, and/or prevents tumor spread or growth compared to the response obtained without administration of the compound. Effective amounts may vary according to factors such as the disease state, age, sex and weight of the animal. The amount of a given compound that will correspond to such an amount will vary depending upon various factors, such as the given drug or compound, the pharmaceutical formulation, the route of administration, the type of disease or disorder, the identity of the subject or host being treated, and the like, but can nevertheless be routinely determined by one skilled in the art.

The term “cancer” as used herein refers to one of a group of diseases caused by the uncontrolled, abnormal growth of cells that can spread to adjoining tissues or other parts of the body. Cancer cells can form a solid tumor, in which the cancer cells are massed together, or exist as dispersed cells, as in leukemia. Cancers affect various tissues and cells. For example cancer includes hematological cancer and solid tumors such as colorectal tumours, hepatocellular carcinoma (HCC), prostate cancer, breast cancer, lung cancer and skin cancer.

The term “precancerous disorder” as used herein refers to one of a group of hyperproliferative disorders that can develop into cancer, including for example precancerous blood disorders, such as monoclonal gammopathy of unknown significance (MGUS) which is a premalignant condition that is related to and/or can develop into MM, and myelodysplastic syndrome (MDS) which is a premalignant condition that is related to and/or can develop into AML.

The term “solid tumor” as used herein refers to an abnormal mass of tissue that typically does not contain liquid areas. Examples include colorectal tumours, prostate cancer, breast cancer, lung cancer and skin cancer as well as sarcomas and carcinomas.

The term “hematological cancer” or “hematological cancer” as used herein refers to cancers that affect blood and bone marrow, and includes without limitation leukemias, lymphomas and multiple myeloma.

The term “hematological precancerous disorder” as used herein means precancerous disorders that affect blood and bone marrow, and includes for example MGUS and MDS.

The term “leukemia” as used herein means any disease involving the progressive proliferation of abnormal leukocytes found in hemopoietic tissues, other organs and usually in the blood in increased numbers. For example, leukemia includes acute myeloid leukemia (AML), acute lymphocytic leukemia (ALL) and chronic myelogenous leukemia (CML).

The term “lymphoma” as used herein means any disease involving the progressive proliferation of abnormal lymphoid cells. For example, lymphoma includes diffuse large B-cell lymphoma, mantle cell lymphoma, Non-Hodgkin's lymphoma, and Hodgkin's lymphoma. Non-Hodgkin's lymphoma would include indolent and aggressive Non-Hodgkin's lymphoma. Aggressive Non-Hodgkin's lymphoma would include intermediate and high grade lymphoma. Indolent Non-Hodgkin's lymphoma would include low grade lymphomas.

The term “myeloma” and/or “multiple myeloma” as used herein means any tumor or cancer composed of cells derived from the hematopoietic tissues of the bone marrow. Multiple myeloma is also knows as MM and/or plasma cell myeloma.

The term “cancer cell” as used herein refers a cell characterized by uncontrolled, abnormal growth and the ability to invade another tissue or a cell derived from such a cell. Cancer cell includes, for example, a primary cancer cell obtained from a patient with cancer or cell line derived from such a cell. Similarly, a “hematological cancer cell” refers to a cancer cell deriving from a blood cell or bone marrow cell.

The term “precancerous cell” as used herein refers a cell characterized by uncontrolled, abnormal growth or a cell derived from such a cell. Precancerous cell includes, for example, a primary precancerous cell obtained from a patient with precancerous disorder or cell line derived from such a cell. Similarly, a “hematological precancerous cell” refers to a precancerous cell deriving from a blood cell or bone marrow cell.

The term “pharmaceutically acceptable” means compatible with the treatment of animals, in particular, humans.

The term “pharmaceutically acceptable salt” means an acid addition salt or a basic addition salt which is suitable for or compatible with the treatment of patients.

The term “pharmaceutically acceptable acid addition salt” as used herein means any non-toxic organic or inorganic salt of any basic compound. Basic compounds that form an acid addition salt include, for example, compound comprising an amine group. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, acid addition salts are more soluble in water and various hydrophilic organic solvents, and generally demonstrate higher melting points in comparison to their free base forms. The selection of the appropriate salt will be known to one skilled in the art.

The term “pharmaceutically acceptable basic addition salt” as used herein means any non-toxic organic or inorganic base addition salt of any acidic compound. Acidic compounds that form a basic addition salt include, for example, compounds comprising a carboxylic acid group. Illustrative inorganic bases which form suitable salts include lithium, sodium, potassium, calcium, magnesium or barium hydroxide. Illustrative organic bases which form suitable salts include aliphatic, alicyclic or aromatic organic amines such as methylamine; trimethylamine and picoline, alkylammonias or ammonia. The selection of the appropriate salt will be known to a person skilled in the art.

The formation of a desired compound salt is achieved using standard techniques. For example, the neutral compound is treated with an acid or base in a suitable solvent and the formed salt is isolated by filtration, extraction or any other suitable method.

The term “prodrug” as used herein refers to a derivative of an active form of a known compound or composition which derivative, when administered to a subject, is gradually converted to the active form to produce a better therapeutic response and/or a reduced toxicity level. In general, prodrugs will be functional derivatives of the compounds disclosed herein which are readily convertible in vivo into the compound from which it is notionally derived. Prodrugs include, without limitation, acyl esters, carbonates, phosphates, and urethanes. These groups are exemplary, and not exhaustive, and one skilled in the art could prepare other known varieties of prodrugs. Prodrugs may be, for example, formed with available hydroxy, thiol, amino or carboxyl groups. For example, the available OH and/or NH₂ in the compounds of the disclosure may be acylated using an activated acid in the presence of a base, and optionally, in inert solvent (e.g. an acid chloride in pyridine). Some common esters which have been utilized as prodrugs are phenyl esters, aliphatic (C₈-C₂₄) esters, acyloxymethyl esters, carbamates and amino acid esters. In certain instances, the prodrugs of the compounds of the disclosure are those in which the hydroxy and/or amino groups in the compounds is masked as groups which can be converted to hydroxy and/or amino groups in vivo. Conventional procedures for the selection and preparation of suitable prodrugs are described, for example, in “Design of Prodrugs” ed. H. Bundgaard, Elsevier, 1985.

The term “solvate” as used herein means a compound or its pharmaceutically acceptable salt, wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”. The formation of solvates will vary depending on the compound and the solvate. In general, solvates are formed by dissolving the compound in the appropriate solvent and isolating the solvate by cooling or using an antisolvent. The solvate is typically dried or azeotroped under ambient conditions.

Where the compounds according to the disclosure possess more than one or more asymmetric centre, they may exist as “stereoisomers”, such as enantiomers and diastereomers. It is to be understood that all such stereisomers and mixtures thereof in any proportion are encompassed within the scope of the present disclosure. It is to be understood that while the stereochemistry of the compounds of the disclosure may be as provided for in any given compound shown herein, such compounds may also contain certain amounts (e.g. less than 20%, less than 10%, less than 5%) of compounds having alternate stereochemistry.

The term “subject” as used herein includes all members of the animal kingdom including mammals, and suitably refers to humans.

The term “treating” or “treatment” as used herein and as is well understood in the art, means an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease (e.g. maintaining a patient in remission), preventing spread of disease, delay or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. “Treating” and “treatment” as used herein also include prophylactic treatment. For example, a subject with early stage myeloma can be treated to prevent progression or alternatively a subject in remission can be treated with a compound or composition described herein to prevent recurrence. Treatment methods comprise administering to a subject a therapeutically effective amount of a compound described herein and optionally consists of a single administration, or alternatively comprises a series of applications. For example, the compounds described herein may be administered at least once a week. However, in another embodiment, the compounds may be administered to the subject from about one time per week to about once daily for a given treatment. In another embodiment, the compound is administered twice daily. The length of the treatment period depends on a variety of factors, such as the severity of the disease, the age of the patient, the concentration, the activity of the compounds described herein, and/or a combination thereof. It will also be appreciated that the effective dosage of the compound used for the treatment or prophylaxis may increase or decrease over the course of a particular treatment or prophylaxis regime. Changes in dosage may result and become apparent by standard diagnostic assays known in the art. In some instances, chronic administration may be required. For example, the compounds are administered to the subject in an amount and for a duration sufficient to treat the patient.

“Palliating” a disease or disorder means that the extent and/or undesirable clinical manifestations of a disorder or a disease state are lessened and/or time course of the progression is slowed or lengthened, as compared to not treating the disorder.

The term “prevention” or “prophylaxis”, or synonym thereto, as used herein refers to a reduction in the risk or probability of a patient becoming afflicted with cancer, for example a reduction in the risk or probability of a patient with a precancerous disorder such as MGUS or MDS, developing MM or AML respectively within a set period of time and/or developing an aggressive form of MM or AML within the set period of time. In addition prevention or prophylaxis can include a reduction in the risk or probability of a patient relapsing. For example, prevention can include use of the compositions described herein for maintenance therapy. For example, after treatment for a primary cancer, a patient in remission may be treated with a composition described herein to prevent recurrence.

### II. Methods

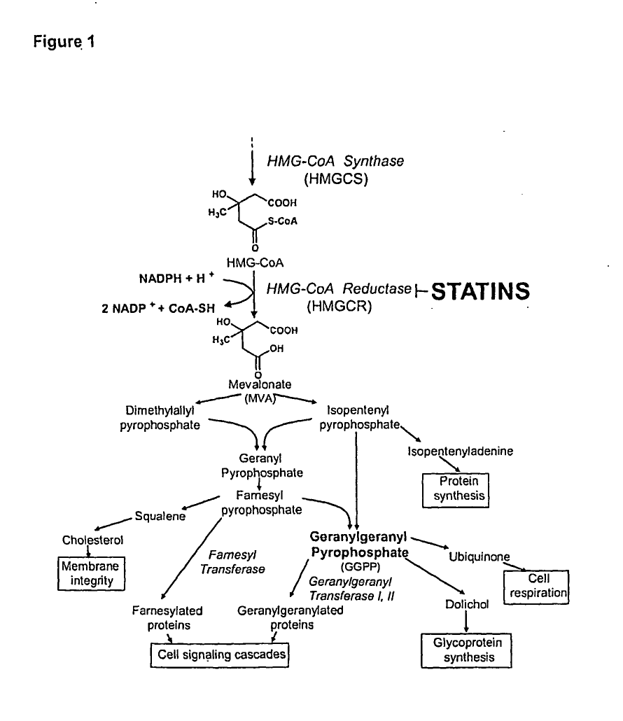

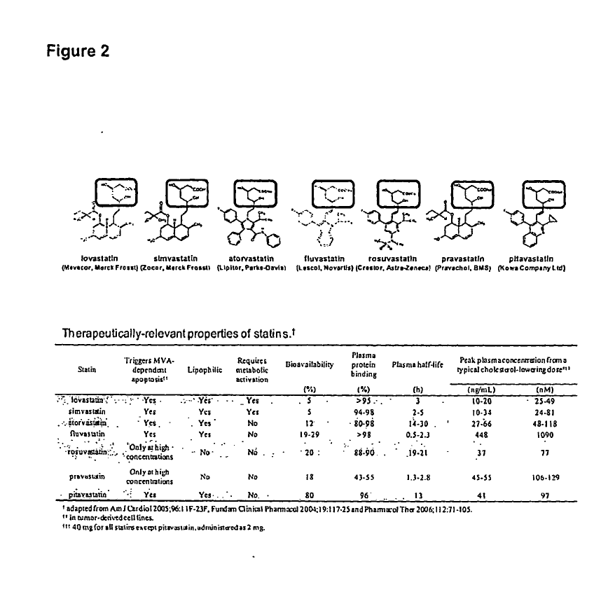

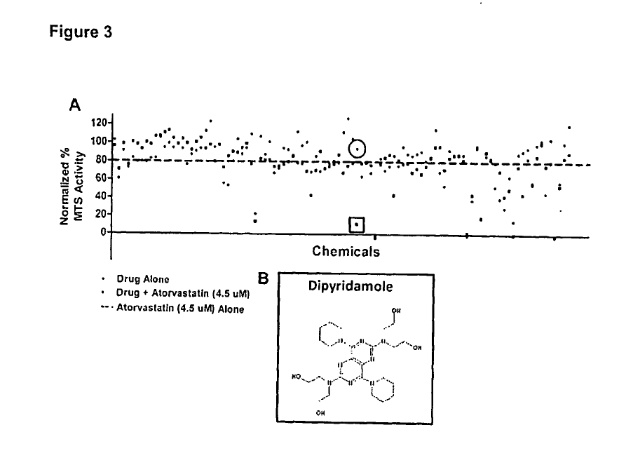

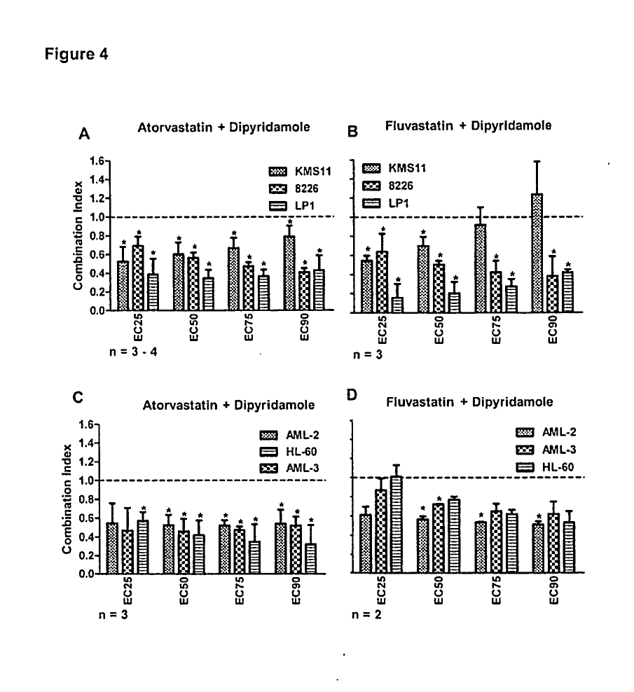

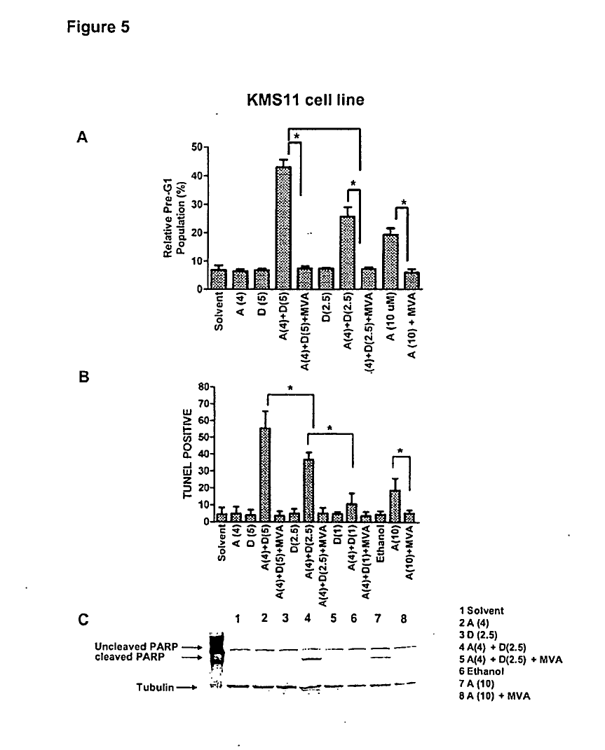

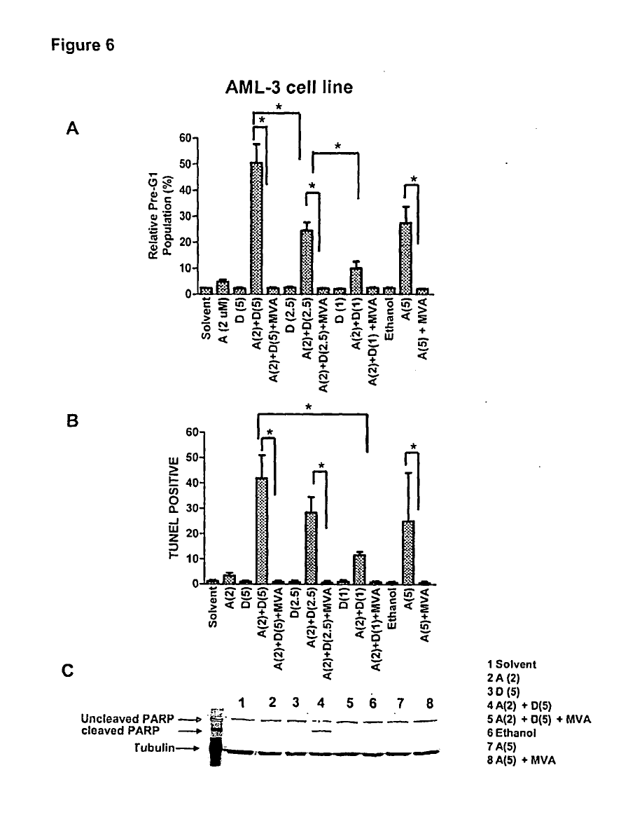

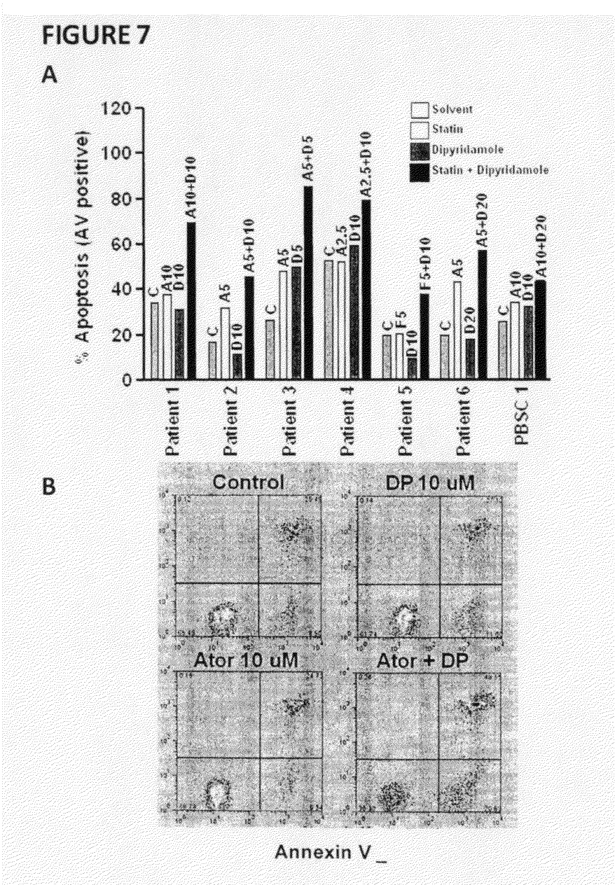

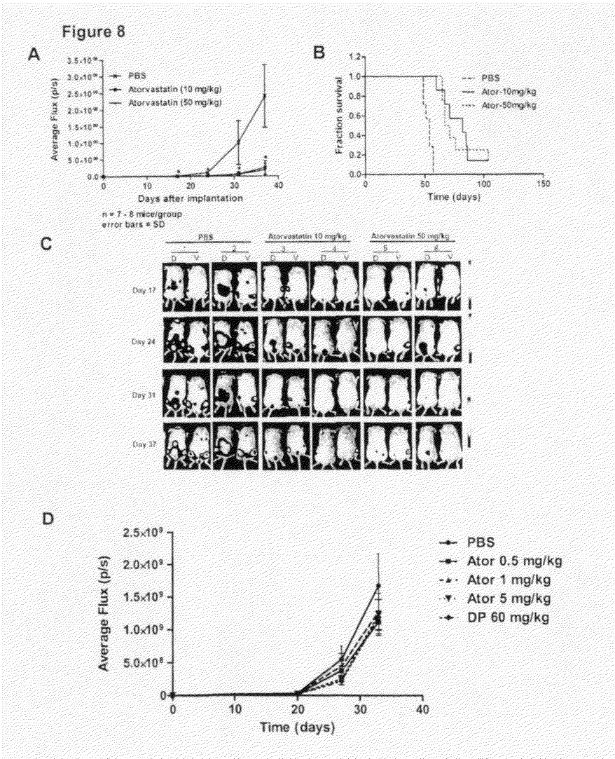

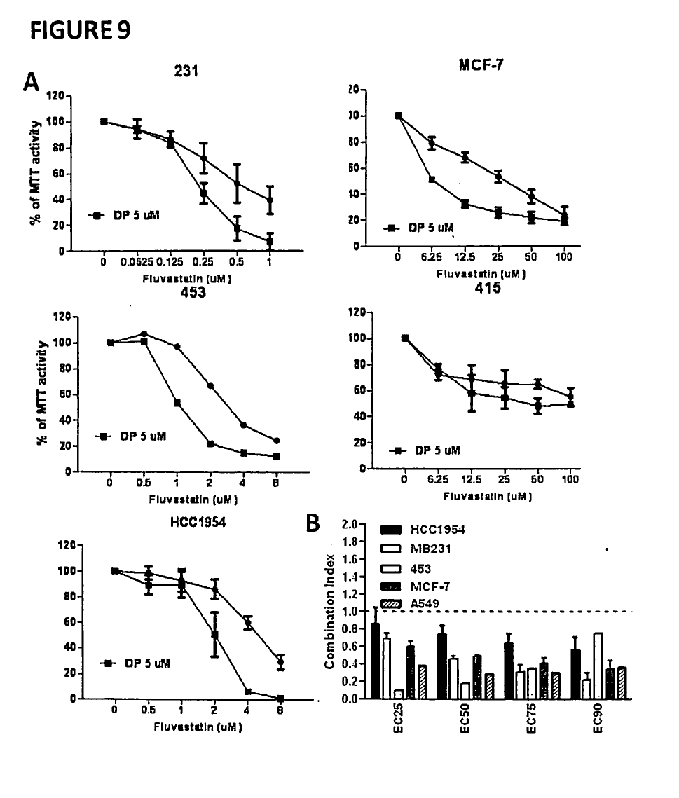

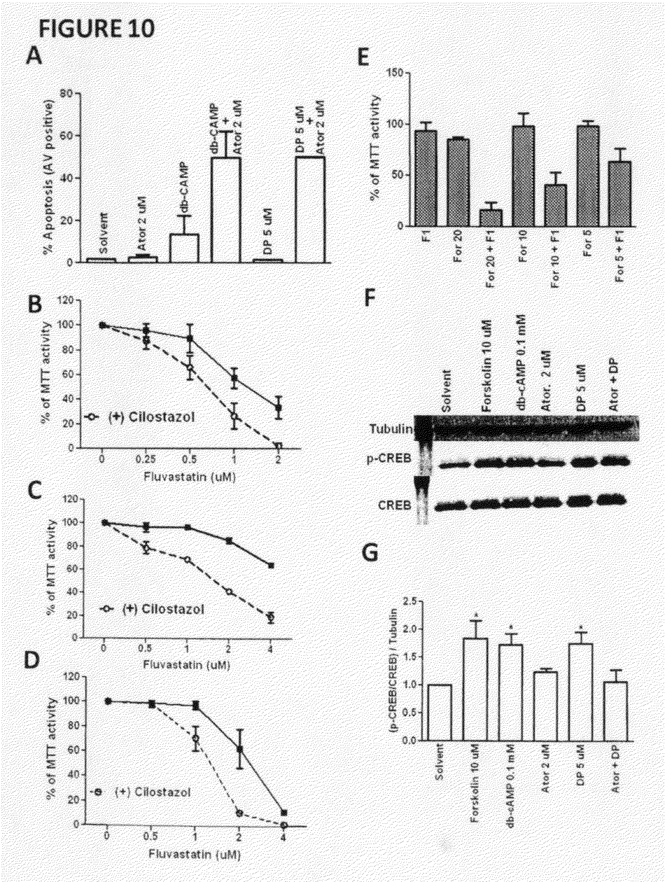

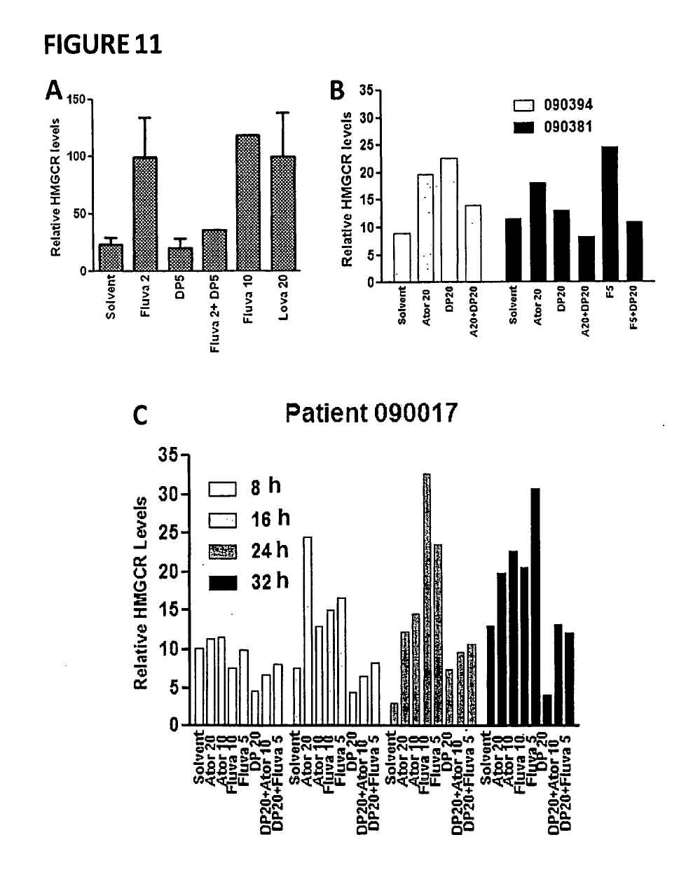

The statin family of drugs target HMGCR, the rate-limiting enzyme of the mevalonate pathway, and have been used for decades in the treatment of hypercholestolemia (FIGS. 1 and 2). It has been shown that statins trigger tumour cells to undergo apoptosis without damaging normal cells. To increase statin efficacy as an anti-cancer agent, a screen was conducted to identify agents that can potentiate atorvastatin-induced apoptosis of tumour cells. Dipyridamole was identified as potentiating the anti-proliferative activity of atorvastatin in a screen conducted on a cell line (KMS11) derived from MM (FIG. 3). Atorvastatin and dipyridamole potentiate and/or synergize in their anti-tumour effects in a variety of cell lines derived from acute myelogenous leukemia (AML) as well as MM (FIGS. 4-6, 9). This potentiation and/or synergy is not restricted to atorvastatin as it is also evident with fluvastatin and dipyridamole (FIGS. 4 and 9), suggesting the statin class of drugs can be used with dipyridamole to potentiate and/or synergistically exhibit anti-tumour effects. This anti-tumour effects occur through the process of apoptosis (FIGS. 5 and 6), suggesting the combination of agents kill tumour cells by a non-inflammatory mechanism of action. Statins (atorvastatin and fluvastatin) and dipyridamole potentiate apoptosis of primary patient AML cells (FIG. 7). Efficacy of atorvastatin alone is shown in an animal xenograft model of MM cells (KMS11) (FIG. 8A-8C). Potentiation is seen also in lung and breast cancer cell lines (FIG. 9) suggesting this combination has potential to be efficacious against all cancers. Further it has been determined that activators of PKA such as adenylate cyclase activators, inhibitors of PDE3, and cell permeable cAMP analogues, can recapitulate the potentiation of cancer cell killing of statins similar to dipyridamole (FIG. 10). Dipyridamole also blocks the upregulation of HMGCR by statin (FIG. 11).

Accordingly, an aspect of the disclosure includes a method of treating a cancer and/or a precancerous disorder comprising administering to a subject in need thereof, an of a statin, in combination with an effective amount of a compound having a dipyridamole activity, optionally dipyridamole and/or a dipyridamole analogue.

In an embodiment, the compound having a dipyridamole activity is a compound that alone or in combination with a statin activates PKA and/or inhibits HMGCR expression.

In another aspect, the disclosure includes a method of preventing cancer and/or reducing a risk of developing cancer comprising administering to a subject in need thereof an effective amount of a statin in combination with an effective amount of a compound having a dipyridamole activity.

In an embodiment, the method comprises administering an effective amount of lovastatin, simvastatin, atorvastatin, fluvastatin, rosuvastatin, pravastatin, or pitavastatin in combination with an effective amount of a compound having dipyridamole activity. In an embodiment, the compound having dipyridamole activity is a PKA activator, and/or a HMGCR expression inhibitor.

In an embodiment, the compound and/or PKA activator is selected from cell permeable cAMP analogues, PDE3 inhibitors, adenylate cyclase activators.

In an embodiment, the compound and/or cell permeable cAMP analogues is db-cAMP or 8-(4-Chlorophenylthio)-2′-O-methylad enosine-3′,5′-cyclic monophosphate sodium salt (8CPT-2Me-cAMP).

In an embodiment, the compound and/or PDE3 inhibitor is selected from cilostazol, aminone or inaminone, milrinone and enoximone.

In an embodiment, the compound and/or adenylate cyclase activator is forskolin.

In an embodiment, the compound is a HMGCR expression inhibitor.

As demonstrated herein, dipyridamole activates PKA and inhibits HMGCR expression. In an embodiment, the compound is PKA activator and a HMGCR expression inhibitor.

In an embodiment, the compound is dipyridamole, and/or a dipyridamole analogue comprising PKA activator activity, and/or HMGCR expression inhibitor activity.

In an embodiment the statin has, within its structure, a moiety that mimics the reaction intermediate formed during the HMGCR catalyzed reaction. In a further embodiment this moiety is a group of the formula Ia or Ib. In a further embodiment, the statin is in the form of a neutral compound or as pharmaceutically acceptable salt. In another embodiment, the statin, or salt thereof, is in the form of a solvate or prodrug thereof. In a further embodiment, the statin is a mixture of two or more statins, or pharmaceutically acceptable salts, solvates or prodrugs thereof. In another embodiment, the statin is selected from lovastatin (Mevacor™), simvastatin (Zocor™) atorvastatin (Lipitor™), fluvastatin (Lescol™), rosuvastatin (Crestor™) pravastatin (Pravachol™), or pitavastatin, or a pharmaceutically acceptable salt, solvate or prodrugs thereof, or a mixture thereof.

In an embodiment, the method comprises administering an effective amount of lovastatin, simvastatin, atorvastatin, fluvastatin, rosuvastatin, pravastatin, or pitavastatin in combination with an effective amount of a compound with dipyridamole activity such as PKA activator activity and/or HMGCR expression inhibitor activity, optionally dipyridamole.

In another embodiment, the method comprises administering an effective amount of lovastatin in combination with an effective amount of dipyridamole. In another embodiment, the method comprises administering an effective amount of atorvastatin in combination with an effective amount of dipyridamole. In another embodiment, the method comprises administering an effective amount of fluvastatin in combination with an effective amount of dipyridamole. In another embodiment, the method comprises administering an effective amount of pitavastatin in combination with an effective amount of dipyridamole. In another embodiment, the method comprises administering an effective amount of simvastatin in combination with an effective amount of dipyridamole. In another embodiment, the method comprises administering an effective amount of rosuvastatin in combination with an effective amount of dipyridamole. In another embodiment, the method comprises administering an effective amount of pravastatin in combination with an effective amount of dipyridamole.

In an embodiment, the effective amount of the statin, for example atorvastatin, is administered before administering the effective amount of dipyridamole and/or compound having dipyridamole activity e.g. PKA activator, PDE3 inhibitor and/or HMGCR expression inhibitor. In another embodiment, the effective amount of the statin, for example atorvastatin, is administered after administering the effective amount of dipyridamole and/or compound having dipyridamole activity. In another embodiment the effective amount of the statin and the effective amount of dipyridamole and/or compound having dipyridamole activity are administered contemporaneously. In an embodiment, the compounds are administered in a single dose or in multiple applications, at similar or different intervals.

In an embodiment, the compounds are administered in amounts that together are sufficient to treat a cancer and/or a precancerous disorder. In another embodiment, the compounds are administered in amounts that together are sufficient to treat a hematological cancer and/or a hematological precancerous disorder.

In an embodiment, the cancer is a hematological cancer, or a solid tumor. In an embodiment the cancer is breast cancer, lung cancer, a colorectal cancer, prostate cancer, skin cancer or hepatocellular carcinoma (HCC). In another embodiment, the cancer is hematological cancer. In an embodiment, the subject is in remission and a composition described herein is administered as maintenance therapy.

In another embodiment, the hematological cancer is a leukemia. In another embodiment, the leukemia is selected from acute myeloid leukemia (AML), acute lymphocytic leukemia (ALL) and chronic myelogenous leukemia (CML).

In a further embodiment, the hematological cancer is a myeloma or MM.

In yet a further embodiment, the hematological cancer is a lymphoma. In an embodiment, the lymphoma is diffuse large B-cell lymphoma. For example, it has been suggested that statins may play a beneficial effect on the outcomes of patients with Diffuse large B cell lymphoma in a prospective observational study. (Nowakowski, 2009, Journal of clinical oncology, 1). In addition, statin treatment of mice with lymphoma showed anti-tumour activity (Shachaf, 2007, Blood, (56).

In another embodiment the precancerous disorder is a hematological precancerous disorder. In a further embodiment, the hematological precancerous disorder is MGUS or MDS.

In an embodiment, compound having dipyridamole activity, such as a PKA activator and/or HMGCR expression inhibitor, significantly increases statin induced cell killing by 20% or more, and significantly shifts the statin dose-response curve and IC50 (the half-maximal inhibitory concentration).

Another embodiment includes a use of an effective amount of statin, in combination with an effective amount of a compound having a dipyradimole activity for the treatment of a cancer and/or a precancerous disorder.

Also disclosed in another embodiment, is use of a statin, in combination with a compound having dipyradimole activity for the manufacture of a medicament for the treatment of a cancer and/or a precancerous disorder.

In an embodiment, the use is for the treatment of a hematological cancer and/or a hematological precancerous disorder. In a further embodiment, the use is for treatment of AML. In yet another embodiment, the use is for the treatment of MM.

Another embodiment includes a use of an effective amount of statin, in combination with an effective amount of dipyradimole and/or compound having dipyridamole activity for the treatment of cancer and/or a precancerous disorder.

Also disclosed in another embodiment, is use of a statin, in combination with dipyradimole and/or compound having dipyridamole activity for the manufacture of a medicament for the treatment of cancer and/or a precancerous disorder.

In an embodiment, the use is for the treatment of a cancer. In an embodiment the use is for treatment of a hematological cancer. In a further embodiment, the use is for treatment of a solid cancer such as breast, lung, prostate, skin or HCC. In a further embodiment, the use is for treatment of AML and/or MM. In another embodiment, the use is for treatment of a precancerous condition. In yet another embodiment, the use is for the treatment of MDS and/or MGUS. In yet a further embodiment, the use is for maintenance therapy.

In another aspect the disclosure includes a method of inducing cell death in a cancer cell and/or in a precancerous cell comprising contacting the cell with a statin in combination with dipyridamole and/or a compound with dipyridamole activity such as a dipyridamole analogue. In an embodiment the cell is a solid cancer cell, a hematological cancer cell or a hematological precancerous cell.

In another embodiment, the method of inducing cell death in a hematological cancer cell and/or precancerous cell comprises contacting the cell with an effective amount of the statin and an effective amount of a compound having PKA activator activity such as dipyridamole and/or dipyridamole analogue.

In an embodiment, the cancer cell is a hematological cancer cell. In an embodiment, the hematological cancer cell is leukemic cell, such as an AML cell, an ALL cell or a CML cell. In an embodiment, the hematological cancer cell is a lymphoma cell. In another embodiment, the hematological cancer cell is a myeloma cell.

In an embodiment, the cancer cell is a solid tumor cell or for example derived from a solid tumor. In an embodiment, the solid tumor cell is a breast cancer cell or a lung cancer cell. In an embodiment, the solid tumor cell is a colorectal cancer cell, a skin cancer cell, a prostate cancer cell or a HCC cell.

In a further embodiment, the precancerous cell is a hematological precancerous cell. In another embodiment, the precancerous cell is a MGUS or a MDS cell.

In an embodiment, the cell is in vitro. In another embodiment, the cell is in vivo.

In an embodiment, the statin is selected from lovastatin, simvastatin, atorvastatin, fluvastatin, rosuvastatin, pravastatin, and pitavastatin.

In an embodiment, compounds in the methods and uses described herein are comprised in a composition, dosage or dosage form described herein.

### III. Compositions

An aspect of the disclosure includes a composition comprising a statin and a compound having a dipyridamole activity such as a PKA activator or a HMGCR expression inhibitor such as dipyridamole and/or a dipyridamole analogue and optionally a suitable carrier or vehicle.

In an embodiment, the composition comprises an effective amount of a statin and an effective amount of PKA activator and/or a HMGCR expression inhibitor and optionally a suitable carrier or vehicle. In an embodiment, the PKA activator, is a cell permeable cAMP analogue, a PDE inhibitor, optionally PDE3 inhibitor, or an adenylate cyclase activator.

In an embodiment, the compound is forskolin, db-cAMP, and/or cilostazol.

In an embodiment, the composition comprises an effective amount of a statin and an effective amount of dipyridamole and/or dipyridamole analogue and optionally a suitable carrier or vehicle.

In another aspect, the composition comprises an effective amount of a statin and an effective amount of a compound having dipyridamole activity such as dipyridamole and/or dipyridamole analogue and optionally a suitable carrier or vehicle for treating a cancer and/or precancerous disorder. In an embodiment, the cancer is a hematological cancer. In an embodiment, the cancer is a solid cancer. In another embodiment, the precancerous disorder is a hematological precancerous disorder.

In a further aspect, the composition comprises an effective amount of a statin and an effective amount of dipyridamole and/or dipyridamole analogue and optionally a suitable carrier or vehicle for preventing and/or reducing the risk of developing cancer in a subject. In an embodiment, the subject is in remission.

In an embodiment, the composition comprises an effective amount of a statin and an effective amount of dipyridamole and/or dipyridamole analogue and a suitable carrier or vehicle for treating a hematological cancer and/or a hematological precancerous disorder.

Another embodiment includes where the composition comprises a statin and a compound having dipyridamole activity for the manufacture of a medicament for the treatment of cancer and/or a precancerous disorder.

In another embodiment, the composition comprises an effective amount of a statin and an effective amount of dipyridamole and/or dipyridamole analogue and a suitable carrier or vehicle for the manufacture of a medicament for the treatment of cancer and/or a precancerous disorder.

In a further embodiment, the precancerous disorder is a hematological precancerous disorder. In yet a further embodiment, the hematological precancerous disorder is MGUS or MDS.

In an embodiment, the cancer is selected from a hematological cancer, and a solid tumor.

In another embodiment, the cancer is selected from colorectal, skin, HCC, prostate cancer, breast, and lung cancer.

In an embodiment, the composition is used for maintaining a subject in remission.

In an embodiment, the cancer is a hematological cancer. In another embodiment, the hematological cancer is a leukemia. In another embodiment, the leukemia is selected from acute myeloid leukemia (AML), acute lymphocytic leukemia (ALL) and chronic myelogenous leukemia (CML).

In a further embodiment, the hematological cancer is a myeloma or MM.

In yet a further embodiment, the hematological cancer is a lymphoma. In an embodiment, the lymphoma is diffuse large B-cell lymphoma.

Yet a further embodiment includes a composition comprising a statin and a dipyridamole and/or dipyridamole analogue for inducing cell death in a cancer cell and/or a precancerous cell.

In an embodiment, the cancer cell is a hematological cancer cell. In another embodiment, the hematological cancer cell is leukemic cell, an AML cell, an ALL cell or a CML cell. In another embodiment, the hematological cancer cell is a myeloma cell. In an embodiment, the hematological cancer cell is a lymphoma cell. In another embodiment, the precancerous cell is a MGUS cell or a MDS cell.

In an embodiment, the compound having dipyridamole activity is selected from a PKA activator compound and/or an HMGCR inhibitor compound. In an embodiment, the compound is a PDE inhibitor such as a PDE3 inhibitor. In an embodiment, the compound is selected from cilostazol, db-cAMP and forskolin.

In an embodiment the statin has, within its structure, a moiety that mimics the reaction intermediate formed during the HMGCR catalyzed reaction. In a further embodiment this moiety is a group of the formula Ia or Ib. In a further embodiment, the statin is in the form of a neutral compound or as pharmaceutically acceptable salt. In another embodiment, the statin, or salt thereof, is in the form of a solvate or prodrug thereof. In a further embodiment, the statin is a mixture of two or more statins, or pharmaceutically acceptable salts, solvates or prodrugs thereof. In an embodiment, the statin is selected from lovastatin, simvastatin, atorvastatin, fluvastatin, rosuvastatin, pravastatin, and pitavastatin. In an embodiment, the statin is lovastatin. In an embodiment, the statin is atorvastatin. In an embodiment, the statin is fluvastatin. In an embodiment, the statin is pitavastatin.

In an embodiment, the dipyridamole and/or dipyridamole analogue is comprised in a composition with acetyl salicylic acid (ASA). Accordingly, in an embodiment, the composition of the present disclosure comprises a statin, dirpyridamole and ASA. In a further embodiment the DP and/or dipyridamole analogue and ASA are formulated in a single composition. In an embodiment, the dipyridamole and/or dipyridamole analogue and ASA are comprised in a tablet or capsule. In an embodiment, the dipyridamole and ASA are provided in a formulation for example, as sold under the brand name Aggrenox™.

In another embodiment, dipyridamole and/or dipyridamole analogue is comprised in an oral dipyridamole formulation (for example comprising 25 mg, 50 mg 75 mg or 100 mg; sold for example as Apo-Dipyridamole-FC (Apotex), Dipyridamol (Sandoz), or by Amide Pharmaceutical, Barr Laboratories, Clonmel Healthcarem, Impax Laboratories, Pro Doc, Purepac Pharmaceuitcals, Zydus Pharmaceuticals). In another embodiment, the dipyridamole and/or dipyridamole analogue is comprised in a suspension, for example 50 mg/ml, or 50 mg/5 ml sold for example by Rosemont Pharmaceuticals) or comprised in a capsule dosage form (for example, a 200 mg capsule, modified and sustatined release, sold as Persantin Retard and Persantin SR respectively, Boehringer Ingelheim). In another embodiment, dipyridamole and/or dipyridamole analogue is comprised in an injectable dipyridamole formulation (e.g. 5 mg/ml, American Pharmaceutical Partners, Apotex, Baxter, Bedford Laboratories, Hospira, Novopharm, Pharmaceutical Partners of Canada, Sicor Pharmaceuticals, Boehringer Ingelheim (IV Persantine), or as a oral suspension comprising for example about 50 mg dipyridamole/5 ml.

The compounds are suitably formulated into pharmaceutical compositions for administration to human subjects in a biologically compatible form suitable for administration in vivo.

The compositions described herein can be prepared by per se known methods for the preparation of pharmaceutically acceptable compositions that can be administered to subjects, such that an effective quantity of the active substance is combined in a mixture with a pharmaceutically acceptable vehicle.

Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition can be supplied, for example but not by way of limitation, as a lyophilized powder which is reconstituted with sterile water or saline prior to administration to the subject.

Suitable pharmaceutically acceptable carriers include essentially chemically inert and nontoxic compositions that do not interfere with the effectiveness of the biological activity of the pharmaceutical composition.

Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesyl-phosphotidyl-ethanolamine (DOPE), and liposomes. Such compositions should contain a therapeutically effective amount of the compound(s), together with a suitable amount of carrier so as to provide the form for direct administration to the subject.

In an embodiment, the compositions described herein are administered for example, by parenteral, intravenous, subcutaneous, intramuscular, intracranial, intraorbital, ophthalmic, intraventricular, intracapsular, intraspinal, intracisternal, intraperitoneal, intranasal, aerosol, topical or oral administration.

Compositions for nasal administration can conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomizing device. Alternatively, the sealed container may be a unitary dispensing device such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Wherein the route of administration is oral, the dosage form may be for example, incorporated with excipient and used in the form of enteric coated tablets, caplets, gelcaps, capsules, ingestible tablets, buccal tablets, troches, elixirs, suspensions, syrups, wafers, and the like. The oral dosage form may be solid or liquid.

In an embodiment, the disclosure describes a pharmaceutical composition wherein the dosage form is a solid dosage form. A solid dosage form refers to individually coated tablets, capsules, granules or other non-liquid dosage forms suitable for oral administration. It is to be understood that the solid dosage form includes, but is not limited to, modified release, for example immediate release and timed-release, formulations. Examples of modified-release formulations include, for example, sustained-release (SR), extended-release (ER, XR, or XL), time-release or timed-release, controlled-release (CR), or continuous-release (CR or Contin), employed, for example, in the form of a coated tablet, an osmotic delivery device, a coated capsule, a microencapsulated microsphere, an agglomerated particle, e.g., as of molecular sieving type particles, or, a fine hollow permeable fiber bundle, or chopped hollow permeable fibers, agglomerated or held in a fibrous packet. Timed-release compositions can be formulated, e.g. liposomes or those wherein the active compound is protected with differentially degradable coatings, such as by microencapsulation, multiple coatings, etc. It is also possible to freeze-dry the compounds described herein and use the lyophilizates obtained, for example, for the preparation of products for injection.

Compositions suitable for buccal or sublingual administration include tablets, lozenges, and pastilles, wherein the active ingredient is formulated with a carrier such as sugar, acacia, tragacanth, and/or gelatin and/or glycerin.

In another embodiment, the disclosure describes a pharmaceutical composition wherein the dosage form is a liquid oral dosage form. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia The National Formulary (USP 24 NF19) published in 1999.

In another embodiment, the disclosure describes a pharmaceutical composition wherein the dosage form is an injectable dosage form. An injectable dosage form is to be understood to refer to liquid dosage forms suitable for, but not limited to, intravenous, subcutaneous, intramuscular, or intraperitoneal administration. Solutions of compounds described herein can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

### IV. Kits and Packs

Another aspect of the disclosure includes a kit. In an embodiment, the kit comprises a statin and instructions for administering in combination with a compound with dipyridamole activity such as dipyridamole and/or dipyridamole analogue. In another embodiment, the kit comprises a statin and/or a compound with dipyridamole activity such as dipyridamole and/or a dipyridamole analogue and instructions for administering in combination with the other.

Another aspect of the disclosure includes a pack e.g. a pharmaceutical pack. The compositions and/or formulations described herein for treating a cancer, such as a solid cancer, a hematological cancer, and/or precancerous disorder, can be comprised in a pharmaceutical pack or vial. In an embodiment, the pharmaceutical pack or vial comprises a statin and/or dipyridamole and/or dipyridamole analogue and instructions for administering with the other for example a daily dose for the treatment of a cancer such as a hematological cancer and/or precancerous disorder.

The following non-limiting examples are illustrative of the present disclosure:

### EXAMPLES

### Example 1

One hundred compounds were evaluated for their ability to potentiate the anti-proliferative activity of atorvastatin on statin-sensitive KMS11 mM cells. Briefly, cells were exposed to sublethal doses of atorvastatin (IC20) alone and in combination with sublethal doses of the compounds.

One compound, dipyridamole (DP), showed remarkable potentiation and was further explored. Statins (atorvastatin and fluvastatin) and DP synergized to exert anti-tumour effects on KMS11 cells as well as a panel of other MM and AML cell lines (FIG. 4). When combined, individually sublethal doses of both atorvastatin and DP, showed a remarkable induction of apoptosis in a dose-dependent manner as assayed by fixed-PI, TUNEL and PARP cleavage in representative MM and AML cell lines (FIGS. 5 and 6). The synergistic effect was reversible with exogenous addition of MVA (FIGS. 5 and 6) Importantly, statins (atorvastatin and fluvastatin) and DP induced apoptosis in AML primary patient samples.

DP (Persantine) is used in the control of recurrent stroke and is widely prescribed as a vasodilator and inhibitor of platelet aggregation. DP has been shown to elicit numerous effects at both the molecular and physiological levels: inhibits phosphodiesterases, which increases intracellular cyclic AMP and GMP; inhibits nucleoside and glucose transport; and possesses antioxidant and anti-inflammatory properties (43-48). The data shows that the chemical screening strategy can successfully identify compounds that potentiate and/or synergize with statins to potentiate apoptosis of tumour cells (49-51).

### Example 2

Atorvastatin treatment, p.o. (oral delivery) three times a week for 37 days initiated 2 days post KMS-11-Iuc injection (8 million cells/mouse inoculated i.v. via the tail) significantly reduced tumor burden as shown in FIG. 8A and prolonged survival as shown in FIG. 8B in the early stage orthotopic MM disease model (n=7-8 mice/group). Tumours were imaged in mice on designated days by whole-body imaging using the IVIS imaging system illustrated by a representative image (C). Briefly, mice were i.p. injected with luciferein followed by anesthetization with isoflurane. Signal intensity was quantified using Living Image versio 2.50.2 (Xenogen) by summing detected photon counts from dorsal and ventral images. Lower doses of atorvastatin (p.o. 3 times a week) as well as DP (p.o. 5 times a week) had no significant effect, (n=3-5 mice/group) alone (D).

Doses of atorvastatin and dipyridamole that are themselves not able to retard tumour growth have been established (FIG. 8D) and these are now undergoing evaluation as combination therapy in the orthotopic MM model described above

### Characterization of the Combination of Atorvastatin and DP to Drive Tumour Cell Apoptosis.

Atorvastatin and DP synergistically trigger apoptosis of tumour cells. To determine whether atorvastatin and DP can block xenograft tumour formation in vivo the effect of treatment of each agent alone and in combination in an early stage orthotopic MM disease model (52) will be evaluated (FIG. 8) Both agents are administered through a common oral route of delivery. These drugs have been titrated and doses of each agent have been established which, when delivered by oral gavage, do not inhibit tumour growth (FIG. 8D). It will be determined whether delivery of both agents in combination will impact tumour growth and animal survival. Efficacy and safety of this drug combination in the first immunocompetant transgenic model of MM (53) will also be assessed. Given that both atorvastatin and DP are approved for use in humans, unacceptable adverse side-effects are not anticipated.

### Example 3

The mechanism of action was investigated. First, to tease apart which of DP's many activities may be functionally critical, the synergistic potential of a panel of agents that mimics DP in one or more of its activities was tested (see Table 1). This approach has been used successfully to distinguish important attributes of other nucleoside inhibitors (63).

TABLE 1
Compounds that share one or more known
activities with dipyridamole

| Compound | Function |
|---|---|
| Cilostazol | Inhibitor of adenosine uptake |
| Dilazep dihydrochloride | Inhibitor of platelet aggregation and of |
| | membrane transport of nucleosides. |
| | Inhibits adenosine uptake. |
| 5-Iodotubercidin | Potent adenosine kinase inhibitor, |
| | nucleoside transporter inhibitor. |
| Fasentin | Inhibitor of glucose transport |
| 4-{[3′,4′-{Methylene- | Potent and specific inhibitor of |
| dioxy)benzyl]amino}-6- | cGMP-specific PDE5. |
| methoxyquinazoline | |
| NBPMR | Potent ENT1 and to a lesser |
| | extent ENT2 inhibitor |
| Zaprinast | Selective PDE5, 6, 9 and 11 inhibitor |

PDE = phosphodiesterase, ENT = equilibrative nucleoside transporter

Only Cilostazol of the above Table 1 compounds potentiated statin cancer cell death (FIG. 10B). As cilostazol inhibits PDE3 which activates. PKA, studies were carried out using various PKA activators, including forskolin, a cell permeable analog of cAMP, db-cAMP, and a PDE3 inhibitor. Addition of a cell-permeable analog of cAMP, dibutyryl-cAMP (db-cAMP 0.25 mM) in combination with atorvastatin recapitulated the DP-atorvastatin apoptotic effects (FIG. 10A) 48 hours post treatment in the AML-3 cell line. Cilostazol, a selective PDE3 inhibitor, also potentiated the cytotoxic effects of statins in AML-3 (FIG. 10B) AML-2 (FIG. 10C), KMS11 (FIG. 10D) cell lines as did forskolin an adenylate cyclase activator in the AML-3 cell line (FIG. 10E) db-cAMP, DP and forskolin increase CREB phosphorylation 2 hours post-treatment in the AML-3 cell line as demonstrated in the representative immunoblot (FIG. 10F) quantified in (FIG. 100) using secondary antibodies labelled with IRDye infrared dyes (LI-COR). n=2-3*p<0.05. Inhibition of PDEs 6, 9 and 11 by zaprinast is expected to raise cGMP but not cAMP levels.

### Example 4

Experiments in breast cancer cell lines (e.g. MCF-7) as well as the lung cancer cell lines (e.g. A549) were carried out similarly as described above.

Dipyridamole potentiated the anti-cancer effects of statins (FIG. 9A) and synergized with statins (FIG. 9B) in several breast cancer and lung cell lines. Drug treatments were performed individually or in a fixed ratio for 72 hours. Synergy was assessed using Chou and Talays combination index. (DP=dipyridamole), n=1-4. Atorvastatin was used in A549's and fluvastatin the breast cancer cell lines to assess potentiation and synergy.

### Example 5

The combination of statins and dipyridamole prevents the upregulation of HMGCR mRNA in LP1 mM cells (FIG. 11A) and primary AML patient samples (FIG. 11B,C). RNA from primary AML and LP1 cells was harvested 24 and 16 hours post compound treatment respectively using TRIZOL reagent. HMGCR mRNA levels were assessed using real-time PCR (SYBR Green). Ator.=Atorvastatin, F or Fluva=Fluvastatin, DP=Dipyridamole, concentrations (μM) are depicted in the bar graphs. For example, Fluva 2 indicates a fluvastatin concentration of 2 μM. Upregulation of HMGCR mRNA in response to statin exposure has been shown to be a distinguishing feature in determining statin-sensitivity in cohorts of statin sensitive and insensitive MM cell lines (Clendening et al., Blood 2010; 115: 4787. Insensitive MM cells had an intact classic feedback response to statin exposure including HMGCR mRNA and protein upregulation while the sensitive cells, this upregulation was lost.

### Example 6

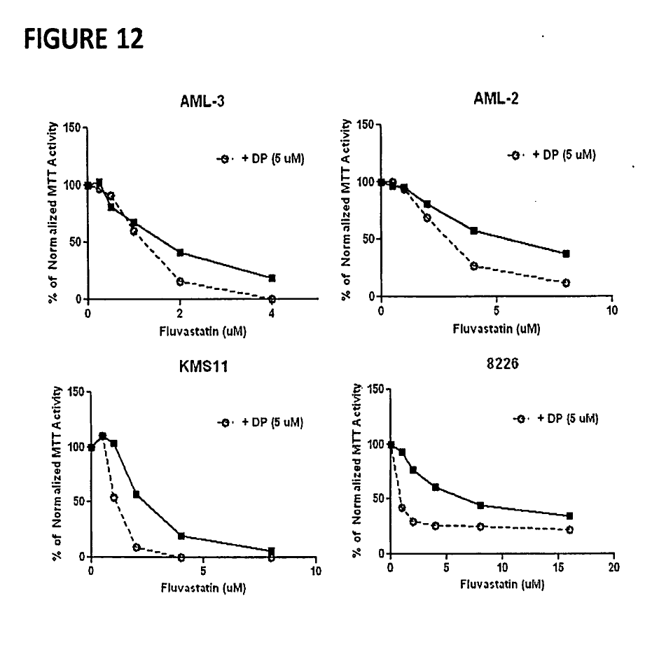

Cilostazol potentiated fluvastatin cell death and the level of potentiation was similar to that seen with dipyridamole+fluvastatin in the AML cell lines and the KMS11 mM cell line (FIG. 12).

### Example 7

PKA activation appears to synergize with statins similar to dipyridamole in AML cells. Dipyridamole activates PKA in terms of inducing CREB phosphorylation. Forskolin, an activator of adenylate cyclase also potentiated the effects of statins in AML cells. It is expected that PKA activators will potentiate statin activity in other cancer cells.

### Example 8

Whether statins in combination with DP can specifically and synergistically target the leukemia stem cell (LSC) in the absence of an apoptotic effect on normal hematopoitic stem cells (HSC) will be determined in vivo. The engraftment of human stem cells derived from human AML or human cord blood into immunocompromised mice is now routinely possible. The primary samples that respond to statins and DP in tissue culture will be advanced to assess the effects of these agents on the ability of primary AML stem cells to engraft immunocompromised mice (54, 55). Statins can target the primitive myeloid progenitor cells from primary AML and not from bone marrow or cord blood, suggesting that statins are able to selectively target the leukemia stem cell (43). Briefly, primary AML or HSC cells will be injected intrafemorally into sublethally irradiated NOD/SCID female mice. Four weeks after injection, mice will be treated by oral gavage with atorvastatin and DP or vehicle control as before (FIG. 8). Mice will then be sacrificed, cells flushed from the femurs, and engraftment of the human cells into the marrow assessed by enumerating the human CD45 cells by flow cytometry. Leukemic cells will be distinguished by the presence of human CD33 and lack of CD19.

While the present disclosure has been described with reference to what are presently considered to be the preferred examples, it is to be understood that the disclosure is not limited to the disclosed examples. To the contrary, the disclosure is intended to cover various modifications and equivalent arrangements included within the spirit, and scope of the appended claims.

All publications, patents and patent applications are herein incorporated by reference in their entirety to the same extent as if each individual publication, patent or patent application was specifically and individually indicated to be incorporated by reference in its entirety.

### FULL CITATIONS FOR REFERENCES REFERRED TO IN THE SPECIFICATION

1. Russell, D. W. (1992) Cholesterol biosynthesis and metabolism. Cardiovasc Drugs Ther 6, 103-110
2. Istvan, E. S., and Deisenhofer, J. (2001) Structural mechanism for statin inhibition of HMG-CoA reductase. Science 292, 1160-1164
3. Endo, A. (1985) HMG-CoA reductase inhibitors. Adv Exp Med Biol 183, 295-310
4. Pedersen, T., and Gaw, A. (2001) Statins—similarities and differences. Am J Manag Care 7, S132-137
5. Vitols, S., Norgren, S., Juliusson, G., Tatidis, L., and Luthman, H. (1994) Multilevel regulation of low-density lipoprotein receptor and 3-hydroxy-3-methylglutaryl coenzyme A reductase gene expression in normal and leukemic cells. Blood 84, 2689-2698
6. Mo, H., and Elson, C. E. (2004) Studies of the isoprenoid-mediated inhibition of mevalonate synthesis applied to cancer chemotherapy and chemoprevention. Exp Biol Med (Maywood) 229, 567-585
7. Friesen, J. A., and Rodwell, V. W. (2004) The 3-hydroxy-3-methylglutaryl coenzyme-A (HMG-CoA) reductases. Genome Biol 5, 248
8. Duncan, R. E., El-Sohemy, A., and Archer, M. C. (2004) Mevalonate promotes the growth of tumors derived from human cancer cells in vivo and stimulates proliferation in vitro with enhanced cyclin-dependent kinase-2 activity. J Biol Chem 279, 33079-33084
9. Gray-Bablin, J., Rao, S., and Keyomarsi, K. (1997) Lovastatin induction of cyclin-dependent kinase inhibitors in human breast cells occurs in a cell cycle-independent fashion. Cancer Res 57, 604-609
10. Wejde, J., Blegen, H., and Larsson, O. (1992) Requirement for mevalonate in the control of proliferation of human breast cancer cells. Anticancer Res 12, 317-324
11. Dale, K. M., Coleman, C. I., Henyan, N. N., Kluger, J., and White, C. M. (2006) Statins and cancer risk: a meta-analysis. JAMA 295, 74-80
12. Boudreau, D. M., Yu, O., Miglioretti, D. L., Buist, D. S., Heckbert, S. R., and Daling, J. R. (2007) Statin use and breast cancer risk in a large population-based setting. Cancer Epidemiol Biomarkers Prev 16, 416-421
13. Poynter, J. N., Gruber, S. B., Higgins, P. D., Almog, R., Bonner, J. D., Rennert, H. S., Low, M., Greenson, J. K., and Rennert, G. (2005) Statins and the risk of colorectal cancer. N Engl J Med 352, 2184-2192
14. Strandberg, T. E., Pyorala, K., Cook, T. J., Wilhelmsen, L., Faergeman, O., Thorgeirsson, G., Pedersen, T. R., and Kjekshus, J. (2004) Mortality and incidence of cancer during 10-year follow-up of the Scandinavian Simvastatin Survival Study (4S). Lancet 364, 771-777
15. Graf, H., Jüngst C, Straub G, Dogan S, Hoffmann R T, Jakobs T, Reiser M, Waggershauser T, Helmberger T, Walter A, Walli A, Seidel D, Goke B, Jüngst D. (2008) Chemoembolization combined with pravastatin improves survival in patients with hepatocellular carcinoma. Digestion 78, 34-8.16. Pocobelli, G., Newcomb, P. A., Trentham-Dietz, A., Titus-Ernstoff, L., Hampton, J. M., and Egan, K. M. (2008) Statin use and risk of breast cancer. Cancer 112, 27-33
17. Bonovas, S., Filioussi, K., Tsavaris, N., and Sitaras, N. M. (2005) Use of statins and breast cancer: a meta-analysis of seven randomized clinical trials and nine observational studies. J Clin Oncol 23, 8606-8612
18. Campbell, M. J., Esserman, L. J., Zhou, Y., Shoemaker, M., Lobo, M., Borman, E., Baehner, F., Kumar, A. S., Adduci, K., Marx, C., Petricoin, E. F., Liotta, L. A., Winters, M., Benz, S., and Benz, C. C. (2006) Breast cancer growth prevention by statins. Cancer Res 66, 8707-8714
19. Taylor, M. L., Wells, B. J., and Smolak, M. J. (2008) Statins and cancer: a meta-analysis of case-control studies. Eur J Cancer Prev 17, 259-268
20. Kumar, A. S., Benz, C. C., Shim, V., Minami, C. A., Moore, D. H., and Esserman, L. J. (2008) Estrogen receptor-negative breast cancer is less likely to arise among lipophilic statin users. Cancer Epidemiol Biomarkers Prev 17, 1028-1033
21. Kuoppala, J., Lamminpaa, A., and Pukkala, E. (2008) Statins and cancer: A systematic review and meta-analysis. Eur J Cancer
22. Cauley, J. A., McTiernan, A., Rodabough, R. J., LaCroix, A., Bauer, D. C., Margolis, K. L., Paskett, E. D., Vitolins, M. Z., Furberg, C. D., and Chlebowski, R. T. (2006) Statin use and breast cancer: prospective results from the Women's Health Initiative. J Natl Cancer Inst 98, 700-707
23. El-Serag, H. B., Johnson, M. L., Hachem, C. & Morgana, R. O. (2009) Statins are associated with a reduced risk of hepatocellular carcinoma in a large cohort of patients with diabetes. Gastroenterology 136, 1601-1608.
24. Kochhar, R., Khurana, V., Bejjanki, H., Caldito, G., and Fort, C. (2005) Statins reduce breast cancer risk: a case control study in US female veterans. J Clin Oncol 23, 514
25. Kwan, M. L., Habel, L. A., Flick, E. D., Quesenberry, C. P., and Caan, B. (2008) Post-diagnosis statin use and breast cancer recurrence in a prospective cohort study of early stage breast cancer survivors. Breast Cancer Res Treat 109, 573-579
26. Thibault, A., Samid, D., Tompkins, A. C., Figg, W. D., Cooper, M. R., Hohl, R. J., Trepel, J., Liang, B., Patronas, N., Venzon, D. J., Reed, E., and Myers, C. E. (1996) Phase I study of lovastatin, an inhibitor of the mevalonate pathway, in patients with cancer. Clin Cancer Res 2, 483-491
27. Kim, W. S., Kim, M. M., Choi, H. J., Yoon, S. S., Lee, M. H., Park, K., Park, C. H., and Kang, W. K. (2001) Phase II study of high-dose lovastatin in patients with advanced gastric adenocarcinoma. Invest New Drugs 19, 81-83
28. Kawata, S., Yamasaki, E., Nagase, T., Inui, Y., Ito, N., Matsuda, Y., Inada, M., Tamura, S., Noda, S., Imai, Y., and Matsuzawa, Y. (2001) Effect of pravastatin on survival in patients with advanced hepatocellular carcinoma. A randomized controlled trial. Br J Cancer 84, 886-891
29. Lopez-Aguilar, E., Sepulveda-Vildosola, A. C., Rivera-Marquez, H., Cerecedo-Diaz, F., Valdez-Sanchez, M., and Villasis-Keever, M. A. (1999) Security and maximal tolerated doses of fluvastatin in pediatric cancer patients. Arch Med Res 30, 128-131
30. Lamer, J., Jane, J., Laws, E., Packer, R., Myers, C., and Shaffrey, M. (1998) A phase I-II trial of lovastatin for anaplastic astrocytoma and glioblastoma multiforme. Am J Clin Oncol 21, 579-583
31. Knox, J. J., Siu, L. L., Chen, E., Dimitroulakos, J., Kamel-Reid, S., Moore, M. J., Chin, S., Irish, J., LaFramboise, S., and Oza, A. M. (2005) A Phase I trial of prolonged administration of lovastatin in patients with recurrent or metastatic squamous cell carcinoma of the head and neck or of the cervix. Eur J Cancer 41, 523-530
32. Lersch, C., Schmelz, R., Erdmann, J., Hollweck, R., Schulte-Frohlinde, E., Eckel, F., Nader, M., and Schusdziarra, V. (2004) Treatment of HCC with pravastatin, octreotide, or gemcitabine—a critical evaluation. Hepatogastroenterology 51, 1099-1103
33. Holstein, S. A., Knapp, H. R., Clamon, G. H., Murry, D. J., and Hohl, R. J. (2006) Pharmacodynamic effects of high dose lovastatin in subjects with advanced malignancies. Cancer Chemother Pharmacol 57, 155-164
34. van der Spek, E., Bloem, A. C., van de Donk, N. W., Bogers, L. H., van der Griend, R., Kramer, M. H., de Weerdt, O., Wittebol, S., and Lokhorst, H. M. (2006) Dose-finding study of high-dose simvastatin combined with standard chemotherapy in patients with relapsed or refractory myeloma or lymphoma. Haematologica 91, 542-545
35. Kornblau, S. M., Banker, D. E., Stirewalt, D., Shen, D., Lemker, E., Verstovsek, S., Estrov, Z., Faded, S., Cortes, J., Beran, M., Jackson, C. E., Chen, W., Estey, E., and Appelbaum, F. R. (2007) Blockade of adaptive defensive changes in cholesterol uptake and synthesis in AML by the addition of pravastatin to idarubicin+high-dose Ara-C: a phase 1 study. Blood 109, 2999-3006
36. Schmidmaier, R., Baumann, P., Bumeder, I., Meinhardt, G., Straka, C., and Emmerich, B. (2007) First clinical experience with simvastatin to overcome drug resistance in refractory multiple myeloma. Eur J Haematol 79, 240-243
37. van der Spek, E., Bloem, A. C., Sinnige, H. A., and Lokhorst, H. M. (2007) High dose simvastatin does not reverse resistance to vincristine, adriamycin, and dexamethasone (VAD) in myeloma. Haematologica 92, e130-131
38. Garwood, E. R., Kumar, A. S., Baehner, F. L., Moore, D. H., Au, A., Hylton, N., Flowers, C. I., Garber, J., Lesnikoski, B. A., Hwang, E. S., Olopade, O., Port, E. R., Campbell, M., and Esserman, L. J. (2009) Fluvastatin reduces proliferation and increases apoptosis in women with high grade breast cancer. Breast Cancer Res Treat
39. Lee, J., Jung, K. H., Park, Y. S., Ahn, J. B., Shin, S. J., Im, S. A., Oh do, Y., Shin, D. B., Kim, T. W., Lee, N., Byun, J. H., Hong, Y. S., Park, J. O., Park, S. H., Lim, H. Y., and Kang, W. K. (2009) Simvastatin plus irinotecan, 5-fluorouracil, and leucovorin (FOLFIRI) as first-line chemotherapy in metastatic colorectal patients: a multicenter phase II study. Cancer Chemother Pharmacol 64, 657-663
40. Wong, W. W., Dimitroulakos, J., Minden, M. D., and Penn, L. Z. (2002) HMG-CoA reductase inhibitors and the malignant cell: the statin family of drugs as triggers of tumor-specific apoptosis. Leukemia 16, 508-519
41. (a) Minden, M. D., Dimitroulakos, J., Nohynek, D., and Penn, L. Z. (2001) Lovastatin induced control of blast cell growth in an elderly patient with acute myeloblastic leukemia. Leuk Lymphoma 40, 659-662 (b) Garwood, E. R., Kumar, A. S., Baehner, F. L., Moore, D. H., Au, A., Hylton, N., Flowers, C.I., Garber J., Lesnikoski, B. A., Hwang, E. S., Olopade, O., Port, E. R., Campbell, M., Esserman, L. J. (2010) Fluvastatin reduces proliferation and increases apoptosis in women with high grade breast cancer. Breast Cancer Res Treat. 119, 37-4.4
42. (http://www.cancergov/clinicaltrials/search) National Cancer Institute (by U.S. National Institutes of Health) Clinical Trials Search.
43. Nelson, J. A., and Drake, S. (1984) Potentiation of methotrexate toxicity by dipyridamole. Cancer Res 44, 2493-2496
44. Shuralyova, I., Tajmir, P., Bilan, P. J., Sweeney, G., and Coe, I. R. (2004) Inhibition of glucose uptake in murine cardiomyocyte cell line HL-1 by cardioprotective drugs dilazep and dipyridamole. Am J Physiol Heart Circ Physiol 286, H627-632
45. Hammond, J. R., Williams, E. F., and Clanachan, A. S. (1985) Affinity of calcium channel inhibitors, benzodiazepines, and other vasoactive compounds for the nucleoside transport system. Can J Physiol Pharmacol 63, 1302-1307
46. Beavo, J. A. (1995) Cyclic nucleotide phosphodiesterases: functional implications of multiple isoforms. Physiol Rev 75, 725-748
47. Komas, N., Le Bec, A., Stoclet, J. C., and Lugnier, C. (1991) Cardiac cGMP-stimulated cyclic nucleotide phosphodiesterases: effects of cGMP analogues and drugs. Eur J Pharmacol 206, 5-13
48. Chakrabarti, S., and Freedman, J. E. (2008) Dipyridamole, cerebrovascular disease, and the vasculature. Vascul Pharmacol 48, 143-149
49. Schimmer, A. D. (2008) Chemical biology—understanding biology and advancing therapy. Clin Invest Med 31, E282-289
50. Xu, G. W., Mawji, I. A., Macrae, C. J., Koch, C. A., Datti, A., Wrana, J. L., Dennis, J. W., and Schimmer, A. D. (2008) A high-content chemical screen identifies ellipticine as a modulator of p53 nuclear localization. Apoptosis 13, 413-422
51. Mao, X., Stewart, A. K., Hurren, R., Datti, A., Zhu, X., Zhu, Y., Shi, C., Lee, K., Tiedemann, R., Eberhard, Y., Trudel, S., Liang, S., Corey, S. J., Gillis, L. C., Barber, D. L., Wrana, J. L., Ezzat, S., and Schimmer, A. D. (2007) A chemical biology screen identifies glucocorticoids that regulate c-maf expression by increasing its proteasomal degradation through up-regulation of ubiquitin. Blood 110, 4047-4054
52. Xin X, Abrams T J, Hollenbach P W, Rendahl K G, Tang Y, Oei Y A, Embry M G, Swinarski D E, Garrett E N, Pryer N K, Trudel S, Jallal B, Mendel D B, Heise C C. (2006) CHIR-258 is efficacious in a newly developed fibroblast growth factor receptor 3-expressing orthotopic multiple myeloma model in mice. Clinical Cancer Research. 12(16):4908-1495
53. Chesi, M., Robbiani, D. F., Sebag, M., Chng, W. J., Affer, M., Tiedemann, R., Valdez, R., Palmer, S. E., Haas, S. S., Stewart, A. K., Fonseca, R., Kremer, R., Cattoretti, G., and Bergsagel, P. L. (2008) AID-dependent activation of a MYC transgene induces multiple myeloma in a conditional mouse model of post-germinal center malignancies. Cancer Cell 13, 167-180
54. Lapidot, T., Sirard, C., Vormoor, J., Murdoch, B., Hoang, T., Caceres-Cortes, J., Minden, M., Paterson, B., Caligiuri, M. A., and Dick, J. E. (1994) A cell initiating human acute myeloid leukaemia after transplantation into SCID mice. Nature 367, 645-648
55. Dick, J. E. (2008) Stem cell concepts renew cancer research. Blood 112, 4793-4807.
56. Shachaf C M, Perez O D, Youssef S, Fan A C, Elchuri S, Goldstein M J, Shirer A E, Sharpe O, Chen J, Mitchell D J, Chang M, Nolan G P, Steinman L, Felsher D W. (2007) Inhibition of HMGcoA reductase by atorvastatin prevents and reverses MYC-induced lymphomagenesis. Blood. 110(7):2674-84.

1. A method of treating a cancer or precancerous disorder comprising administering to a subject in need thereof: 1) a statin, and 2) a compound that increases cAMP levels.

2.-3. (canceled)

4. The method of claim 1, wherein the statin is selected from a compound of Formula Ia or Ib and/or mixtures thereof, optionally selected from lovastatin, simvastatin, atorvastatin, fluvastatin, rosuvastatin, pravastatin, and pitavastatin.

5. The method of claim 1, comprising administering:
lovastatin in combination with dipyridamole and/or a dipyridamole analogue;
atorvastatin in combination with dipyridamole and/or a dipyridamole analogue;
fluvastatin in combination with dipyridamole and/or a dipyridamole analogue; and/or
simvastatin in combination with dipyridamole and/or a dipyridamole analogue.

6.-8. (canceled)

9. The method of claim 1, wherein the compound that increases cAMP levels is selected from dipyridamole, a dipyridamole analogue, a phophodiester (PDE inhibitor and an adenyl cyclase activator.

10. (canceled)

11. The method of claim 9, wherein the compound is selected from dipyridamole, cilostazol, forskolin and/or a cell permeable cAMP analogue.

12.-14. (canceled)

15. The method of claim 11, wherein the cell permeable cAMP analogue is db-cAMP

16. The method of claim 1, wherein the cancer is selected from a hematological cancer or a solid tumor, such as colorectal, prostate, skin, HCC, breast or lung cancer and/or the precancerous disorder is a hematological precancerous disorder.

17. (canceled)

18. The method of claim 16, wherein the hematological cancer is a leukemia, myeloma or lymphoma and/or the hematological precancerous disorder is MGUS or MDS.

19. The method of claim 18, wherein the leukemia is selected from acute myeloid leukemia (AML), acute lymphocytic leukemia (ALL) and chronic myelogenous leukemia (CML).

20.-24. (canceled)

25. The method of claim 1, wherein the subject is in remission.

26. A method according to claim 1 reducing a risk of developing cancer, such as a hematological cancer, comprising administering to a subject in need thereof a statin in combination with a compound that increases cAMP levels.

27. The method of claim 26, wherein the subject has a precancerous disorder, such as a hematological precancerous syndrome such as MGUS or MDS and/or wherein the subject is in remission.

28. (canceled)

29. A composition comprising: 1) a statin and 2) a compound that increases cAMP levels and a suitable carrier or vehicle for treating a cancer or precancerous syndrome and/or for reducing a risk of developing a cancer.

30.-31. (canceled)

32. The composition of claim 29, wherein the compound that increases cAMP levels is selected from dipyridamole analogue a PDE inhibitor such as a PDE3 inhibitor such as cilostazol, an adenylate cyclase activator such as forskolin, and/or a cell permeable cAMP analogue such as db-cAMP.

33. The composition of claim 29, wherein the statin is selected from a statin having of Formula Ia or Ib or mixtures thereof, optionally lovastatin, simvastatin, atorvastatin, fluvastatin, rosuvastatin, pravastatin, and/or pitavastatin.

34.-35. (canceled)

36. The composition of claim 29, wherein the cancer is a hematological cancer, optionally is a leukemia and/or the precancerous syndrome is a hematological precancerous disorder, optionally MGUS or MDS.

37. The composition of claim 36, wherein the leukemia is selected from acute myeloid leukemia (AML), acute lymphocytic leukemia (ALL) and chronic myelogenous leukemia (CML).

38.-40. (canceled)

41. The composition of claim 29, wherein the composition is formulated in a dosage form for oral, parenteral, topical, or intravenous administration.

42. A kit for use in a method of claim 1 comprising a statin and instructions for administering in combination with a compound that increases cAMP levels.

43. The kit of claim 42 comprising the statin and a compound that increases cAMP levels and instructions for administering in combination with the other.

44. A pack for use in a method of claim 1 comprising a statin and dipyridamole and/or a compound that increases cAMP levels and instructions for administering for example a daily dose for the treatment of a cancer such as a hematological cancer or a precancerous disorder such as MGUS of MDS.

45. The method of claim 1, wherein the statin and/or compound that increases cAMP levels is a HMGCR expression inhibitor.
